(19) United States
(12) Patent Application Publication
(10) Pub. No.: US 2005/0192604 A1
(43) Pub. Date: Sep. 1, 2005

(54) Methods and devices for placing a conduit in fluid communication with a target vessel and a source of blood

(76) Inventors: Dean F. Carson, Mountain View, CA (US); Michael L. Reo, Redwood City, CA (US); Stephen L. Olson, Sunnyvale, CA (US); Keke Lepulu, Redwood City, CA (US); Sam T. Crews, Redwood City, CA (US); David H. Cole, Cupertino, CA (US); Adam Sharkawy, Redwood City, CA (US); Darin C. Gittings, Sunnyvale, CA (US); Mark J. Foley, Menlo Park, CA (US)

Correspondence Address: HOEKENDIJK & LYNCH, LLP, P.O. BOX 4787, BURLINGAME, CA, 94011-4787, US

(21) Appl. No.: 11/041,088

(22) Filed: Jan. 21, 2005

Related U.S. Application Data
(63) Continuation of application No. 09/547,532, filed on Apr. 12, 2000, now abandoned.
Application No. 09/547,532 is a continuation-in-part of application No. 09/393,130, filed on Sep. 10, 1999, now abandoned.
Application No. 09/393,130 is a continuation-in-part of application No. 09/232,103, filed on Jan. 15, 1999, now abandoned.
(63) Continuation-in-part of application No. 09/232,062, filed on Jan. 15, 1999, now abandoned.
(63) Continuation-in-part of application No. 09/023,492, filed on Feb. 13, 1998, now abandoned.

(51) Int. Cl. A61N 5/00; A61B 17/08

(52) U.S. Cl. 606/153

(57) ABSTRACT

Devices and methods for placing a conduit in fluid communication with a target vessel to communicate the target vessel with a source of blood. A conduit is coupled to the target vessel by first and second securing components that compress or sandwich the vessel wall. The conduit may be preshaped to assume a desired orientation when in an unbiased state, for example, to allow the conduit to be deformed during delivery and then regain its desired orientation once which is regained when deployed. The first and second securing components may be any shape but are preferably elongated in the direction of the vessel axis, e.g., elliptical or rectangular, such that a minimum amount of material is present at the outlet to closely approximate the cross-sectional area of the native target vessel. The securing components do not significantly occlude the target vessel lumen, may be secured to the vessel wall in non-penetrating fashion, and provides a fluid-tight seal around the attachment site. The conduit may comprise tissue, synthetic material, etc., and one or both securing components may be constructed or provided with means for attaching an autologous vessel.

### CROSS-REFERENCE TO RELATED APPLICATIONS

This application is a continuation-in-part of application Ser. No. 09/393130, filed on Sep. 10, 1999 and entitled “Anastomotic Methods and Devices For Placing a Target Vessel in Fluid Communication with a Source of Blood,” which is a continuation-in-part of application Ser. No. 09/232,103, filed on Jan. 15, 1999 and entitled “Methods and Devices for Forming Vascular Anastomoses,” and application Ser. No. 09/232,062, filed on Jan. 15, 1999 and entitled “Methods and Devices For Bypassing an Obstructed Target Vessel by Placing the Vessel in Communication with a Heart Chamber Containing Blood.” This application is also a continuation-in-part of application Ser. No. 09/023,492, filed on Feb. 13, 1998 and entitled “Methods and Devices Providing Transmyocardial Blood Flow to the Arterial Vascular System of the Heart.” The entire subject matter of each of these parent applications is incorporated herein by reference.

### BACKGROUND OF THE INVENTION

1. Field of the Invention

The invention relates generally to methods and device for placing a conduit in fluid communication with a target vessel and a source of blood, and more particularly, methods and devices for revascularizing the heart by placing the conduit in fluid communication with a coronary vessel, such as a coronary artery or coronary vein, and a source of blood, such as a heart chamber or the aorta.

2. Description of Related Art

Despite the considerable advances that have been realized in cardiology and cardiovascular surgery, heart disease remains the leading cause of death throughout much of the world. Coronary artery disease, or arteriosclerosis, is the single leading cause of death in the United States today. As a result, those in the cardiovascular field continue to search for new treatments and improvements to existing treatments.

Coronary artery disease is currently treated by interventional procedures such as percutaneous transluminal coronary angioplasty (PTCA), coronary stenting and atherectomy, as well as surgical procedures including coronary artery bypass grafting (CABG). The goal of these procedures is to reestablish or improve blood flow through occluded (or partially occluded) coronary arteries, and is accomplished, for example, by enlarging the blood flow lumen of the artery or forming a bypass that allows blood to circumvent the occlusion. What procedure(s) is used typically depends on the severity and location of the blockage. When successful, these procedures restore blood flow to myocardial tissue that had not been sufficiently perfused due to the occlusion.

The improvement and refinement of existing treatments and the search for new treatments are indicative of the significant effort that continues to be expended in order to develop better and more efficient ways of revascularizing the heart. One relatively recently developed treatment, transmyocardial revascularization (TMR), forms small channels in the myocardium so that blood flows directly from the left ventricle to the myocardial tissue. TMR procedures are currently used to treat end-stage patients having limited or no treatment options.

Another proposed treatment places the target vessel, e.g., a coronary artery, in direct fluid communication with a heart chamber containing blood, for example, the left ventricle. Blood flows from the ventricle into a conduit that is in fluid communication with the artery; as such, this treatment may be described as a ventricular bypass procedure. Benefits of this procedure include obviating the need to manipulate the aorta, for example, as is done when a side-biting clamp is used in a typical CABG procedure to create a proximal anastomosis between the bypass graft and the aorta. Clamping or otherwise manipulating the aorta places the patient at risk in some cases due to the likelihood that such manipulation will release embolic material into the bloodstream. Challenges associated with this procedure include delivering and deploying the conduit in the patient's body, properly positioning the conduit with respect to the heart chamber and the target vessel, and obtaining beneficial flow characteristics through the conduit and the target vessel.

A drawback associated with CABG and some ventricular bypass procedures is the harvesting of autologous vessels for use as bypass grafts. Certain patients have no or a limited number of available autologous conduit, for example, due to peripheral vascular diseases. As a result, those in the art have sought to develop synthetic grafts that may be substituted for autologous conduits. Although such synthetic grafts have been somewhat effective when used to treat peripheral vessels, they have not been successful in treating small diameter vessels, such as coronary arteries.

A particularly challenging task that must be performed during CABG procedures, as well as proposed ventricular bypass procedures, is attaching the conduit to the target vessel, particularly when the attachment is performed via a handsewn, sutured anastomosis. Sewing the conduit to the target vessel is a very technical and time-consuming procedure given the diameter of the conduit and the coronary artery, typically from 1 mm to 4 mm. Non-cardiovascular applications, for example, treating peripheral vascular disease or injury, creating arteriovenous fistulas, etc., also typically require the creation of a sutured anastomosis. The difficulty in forming the sutured anastomosis is exacerbated when access to the target vessel is restricted or limited, as is the case in a minimally invasive or percutaneous procedure.

While those in the art have proposed various anastomotic couplings intended to replace a sutured anastomosis, none has performed well enough to receive any level of acceptance in the field. Many of the proposed couplings penetrate or damage the target vessel wall, fail to produce a fluid-tight seal between the conduit and vessel, or are simply cumbersome and difficult to deliver or deploy.

Accordingly, there is a need in the art for improved methods and devices for revascularizing the heart, preferably without manipulating the aorta, as is there a need for an anastomotic coupling that can be used to replace a sutured anastomosis without compromising the quality of the attachment or damaging the vessel being treated. There also remains a need in the art for synthetic conduits suitable for use in both cardiovascular and non-cardiovascular applications. Finally, it would be preferable if such devices and methods, anastomotic couplings and synthetic conduits were designed to be used in a relatively quick, easy and repeatable manner.

### SUMMARY OF THE INVENTION

In one aspect, the invention provides methods and devices for placing a conduit in fluid communication with a target vessel and a source of blood, wherein the conduit is secured to the target vessel and/or the blood source by an anastomotic coupling. In another aspect, the invention provides methods and devices for revascularizing the heart by placing a target vessel in fluid communication with a blood source. The blood source may be a coronary artery or vein, the aorta, a heart chamber, a peripheral vessel, etc.

Revascularization of the heart may be performed via a ventricular bypass procedure carried out according to one embodiment of the invention. This procedure provides several benefits. For example, no aortic manipulation is necessary because a heart chamber is the blood source. Obviating the need to manipulate the aorta significantly reduces stroke risk as well as overall patient morbidity. Also, if an autologous vessel is used to form a conduit for the ventricular bypass procedure, a shorter length is needed than in conventional CABG procedures. This is because the distance between the coronary vessel and the heart chamber is considerably less than the distance between the coronary vessel and the aorta. As a result, a given length of autologous tissue will provide more bypass conduits suitable for use in a ventricular bypass procedure carried out according to the invention.

One embodiment of the invention provides a device for placing a target vessel in fluid communication with a source of blood. The device includes a conduit having a length and a lumen adapted to deliver blood from a blood source to a lumen of a target vessel, a first securing component configured to engage an inner surface of a wall of the target vessel and a second securing component configured to engage an outer surface of the target vessel wall. The first and second securing components are configured to at least partially capture the target vessel wall adjacent an incision in the target vessel wall, and the conduit extends away from the second securing component without passing through the incision in target vessel wall.

Another embodiment of the invention provides a device for placing a target vessel in fluid communication with a source of blood, the device including a conduit adapted to deliver blood from a blood source to a lumen of a target vessel, and first and second securing components respectively configured to engage inner and outer surfaces of a wall of the target vessel adjacent an incision formed therein. The first and second securing components include a tissue-capturing mechanism that at least partially captures tissue of the target vessel wall, and the conduit is coupled to one of the first and second securing components and is secured to the target vessel wall via the tissue-capturing mechanism. The mechanism is configured to substantially fix the relative position of the first and second securing components without penetrating the target vessel wall tissue other than forming the incision in the target vessel wall.

Another embodiment of the invention provides a device for placing a target vessel in fluid communication with a source of blood. The device includes first and second securing components respectively sized and configured to engage the interior and exterior surfaces of the wall of the target vessel, thereby compressing the target vessel wall tissue. A conduit having a length and a lumen adapted to deliver blood from a blood source to the target vessel is coupled to at least one of the first and second securing components by a flexible connection that allows the conduit to be moved with respect to the component.

Another embodiment of the invention provides a device for placing a target vessel in fluid communication with a source of blood, the device including first and second securing components respectively configured to engage at least portions of interior and exterior surfaces of a wall of the target vessel, and a conduit having a length and a lumen adapted to deliver blood from a blood source to a target vessel. The conduit is coupled to at least one of the first and second securing components, and at least part of the conduit is formed in a predetermined shape so as to assume a desired orientation with respect to the target vessel when placed in communication with the source of blood.

Another embodiment of the invention provides a device for placing a target vessel in fluid communication with a source of blood. The device comprises first and second securing components sized and configured to engage the interior and exterior surfaces of the wall of a target vessel. A conduit has a lumen and is adapted to pass through an incision formed in the target vessel wall to deliver blood from a blood source to the target vessel, whereby the conduit and one of the first and second securing components form a blood flow path defined by a continuous surface substantially free of discontinuities to promote desired fluid dynamics through the conduit.

Another embodiment of the invention provides a conduit for placing a target vessel in fluid communication with a source of blood, in combination with a delivery device for use in placing the conduit in a patient's body. The conduit has a length and an inner lumen adapted to deliver blood from a blood source to a target vessel, and is coupled to at least one of first and second securing components. The first securing component is sized and configured to engage an interior surface of a wall of the target vessel while the second securing component is sized and configured to engage an exterior surface of the target vessel wall to capture the target vessel wall between the first and second securing components. A delivery device has a working end for releasably retaining at least one of the first and second securing components.

Another embodiment of the invention provides a method for securing a conduit to a target vessel of a patient's vascular system using steps of providing a conduit adapted to be placed in fluid communication with a lumen of a target vessel, the conduit being coupled to at least one of first and second securing components respectively configured to engage interior and exterior surfaces of a wall of the target vessel adjacent an incision therein, positioning the first securing component through an incision in the target vessel wall and at least partially in the target vessel lumen against the interior surface of the target vessel wall, and positioning the second securing component against the exterior surface of the target vessel wall. The first and second securing components are coupled to secure the conduit to the target vessel wall and create a substantially fluid tight seal between the conduit and the target vessel wall, and the incision is the only penetration formed in the target vessel wall.

Another embodiment of the invention provides a method for using a conduit to place a target vessel of a patient's vascular system in fluid communication with a source of blood. This method includes steps of providing a conduit having one portion adapted to be placed in fluid communication with a source of blood and another portion adapted to be secured to a target vessel, the conduit being configured to assume a first orientation when in a unbiased state. The conduit is biased to a second orientation that is different from the first orientation, secured to the target vessel, and allowed to assume the first orientation with respect to the target vessel.

Another embodiment of the invention provides a method for securing a conduit to a target vessel of a patient's vascular system including steps of providing a conduit coupled to at least one of first and second securing components respectively configured to engage interior and exterior surfaces of a target vessel wall adjacent an incision in the target vessel wall, and engaging a working end of a delivery device with at least a portion of the first securing component to support and manipulate the securing component. At least a part of the first securing component is positioned in a lumen of the target vessel against the interior surface of the target vessel wall, the second securing component is positioned against the exterior surface of the target vessel wall to secure the conduit to the target vessel, and the working end of the delivery device is disengaged from the first securing component.

### BRIEF DESCRIPTION OF THE DRAWING FIGURES

Other features, aspects, benefits and advantages of the invention will be better understood from the following detailed description of preferred embodiments taken in conjunction with the accompanying drawing figures, wherein:

FIG. 1A is a perspective view of a conduit and delivery device constructed according to one embodiment of the invention for placing a target vessel in fluid communication with a source of blood, the conduit including first and second securing components configured to be secured to the target vessel wall;

FIG. 1B is an enlarged view of a distal portion of the delivery device shown in FIG. 1A;

FIG. 2A is a sectional view of one of the securing components and the delivery device shown in FIG. 1A, the delivery device being shown in a conduit-releasing position;

FIG. 2B is a sectional view of the securing component and delivery device shown in FIG. 2A, the delivery device being shown in a conduit-retaining position;

FIG. 3A is a sectional view of the conduit shown in FIG. 1A illustrating the delivery device being used to position one of the securing components in the target vessel;

FIG. 3B is a sectional view of the conduit shown in FIG. 3A illustrating the other securing component being moved into engagement with the target vessel wall to capture the vascular tissue;

FIG. 3C is a sectional view of the conduit shown in FIG. 3B after the delivery device has been moved from the position shown in FIG. 3B to a conduit-releasing position;

FIG. 3D is a sectional view illustrating the delivery device being removed from the conduit shown in FIG. 3C;

FIG. 4A is a perspective view of a portion of a patient's heart with the conduit shown in FIGS. 3A-3D deployed between a coronary vessel and a heart chamber containing blood, wherein the conduit is configured to assume a desired profile with respect to the heart wall;

FIG. 4B is an end elevation view, in section, of the conduit and the portion of the heart shown in FIG. 4A;

FIG. 5A is a perspective view of a conduit constructed according to another embodiment of the invention, wherein the conduit comprises a separate member attached to one of the securing components, and the delivery device shown in FIGS. 2A-2B is used to position the other securing component in the target vessel;

FIG. 5B is a sectional view of the conduit and delivery device shown in FIG. 5A with the securing components moved into engagement with the target vessel wall to capture the vascular tissue;

FIG. 6A is a perspective view of a portion of a conduit constructed according to another embodiment of the invention for placing a target vessel in fluid communication with a source of blood, wherein the conduit is being attached to target vessel;

FIG. 6B is a perspective view of the conduit portion shown in FIG. 6A positioned to communicate with the target vessel;

FIG. 7A is a perspective view of the conduit shown FIGS. 6A and 6B configured for use in a ventricular bypass procedure;

FIG. 7B is a perspective view of the conduit shown in FIG. 7A positioned to communicate a coronary artery with the left ventricle;

FIG. 8 is a perspective view of a portion of a conduit constructed according to another embodiment of the invention;

FIGS. 9A and 9B are perspective views of first and second securing components comprising an attachment portion of the conduit;

FIGS. 10A-10C, respectively, are plan, front and end elevation views of one of the securing components shown in FIG. 9A;

FIGS. 11A and 11B are perspective views sequentially illustrating an exemplary means being used to attach a conduit to one of the securing components of the conduit attachment device;

FIG. 12 is a perspective view of an alternative conduit constructed according to the invention;

FIG. 13 is a perspective view of a conduit component constructed according to another embodiment of the invention;

FIGS. 14 and 15 show alternative conduit configurations in connection with a ventriculocoronary bypass procedure;

FIG. 16A is a side elevation view, partially in section, showing a conduit constructed to another embodiment of the invention in a first position;

FIGS. 16B and 16C are, respectively, side elevation views of the conduit shown in FIG. 1 3A in second and third positions;

FIG. 17A is a perspective view showing a conduit constructed according to the invention in a disassembled, tissue-releasing position;

FIG. 17B is a perspective view showing the conduit of FIG. 17A in an assembled, tissue-capturing position;

FIGS. 18A and 18B are perspective views of conduit securing components constructed according to other embodiments of the invention;

FIGS. 19A and 19B are, respectively, perspective and transverse sectional views of a malleable conduit constructed according to the invention;

FIGS. 19C and 19D are, respectively, perspective and transverse sectional views of a malleable conduit constructed according to an alternative embodiment of the invention;

FIG. 20A is a perspective view showing a conduit constructed according to another embodiment of the invention in a tissue-releasing position;

FIGS. 20B is a sectional view taken through one of the securing components of the conduit shown in FIG. 20A;

FIGS. 20C is a sectional view showing the conduit of FIG. 20A with the securing components in a tissue-capturing position;

FIGS. 21A-21C are perspective views of conduits constructed according to additional embodiments of the invention;

FIG. 22 is a perspective view of a patient with ports formed in the patient's chest wall to access the heart;

FIG. 23A is a sectional view of the patient's chest cavity shown in FIG. 22 including the ports, wherein the lateral aspect of the heart is visible for use in illustrating an exemplary application of another embodiment of the invention;

FIGS. 23B-23F are sequential views similar to FIG. 23A showing a conduit constructed according to the invention being placed in the patient's heart pursuant to a ventricular bypass procedure; and

### DETAILED DESCRIPTION OF PREFERRED EMBODIMENTS

The present invention relates to methods and devices for securing a conduit to a target vessel, as well as methods and devices for placing the conduit in fluid communication with a source of blood. Various conduit configurations, anastomotic couplings for securing the conduit to the target vessel or the blood source, and methods for establishing one ore more flow paths between the blood source and the target vessel are disclosed as well.

In a preferred embodiment, the conduit is coupled to a source of blood, for example, a heart chamber containing oxygenated blood, and a target vessel, for example, a coronary vessel (e.g., artery or vein). It will be recognized, however, that the invention may be used to form a blood flow path between any other luminal structures, some examples of which are set forth below. As used herein, luminal structure means any anatomical structure, natural or synthetic, that is hollow and defines a lumen, for example, a blood vessel or tubular organ. Also, as used herein, source of blood refers to any blood-containing or blood-supplying structure, while oxygenated blood refers to blood that contains some level of oxygen.

The lumen of the target vessel being treated may be partially or completely obstructed by an occlusion, with the conduit placed to form a blood flow path that bypasses the occlusion. Alternatively or additionally, the conduit may be used to create a supplemental blood flow path that feeds into the target vessel to augment blood flow (native or other) already present in the vessel.

The conduit of the invention may be configured in various manners. In its most preferred form, the conduit includes a body and an attachment portion that is secured to the target vessel wall to form an anastomotic connection between the conduit and the vessel. The attachment portion may be secured to the target vessel wall by various means that achieve a secure, sealed attachment, preferably via a tight seal against the tissue of the vessel wall. The preferred attachment portion includes first and second securing components that move between tissue-capturing and tissue-releasing positions to form the connection. The most basic attachment portion according to the invention comprises merely preparing a portion of the conduit, e.g., an autologous vessel, for attachment to the target vessel via a hand-sewn anastomosis.

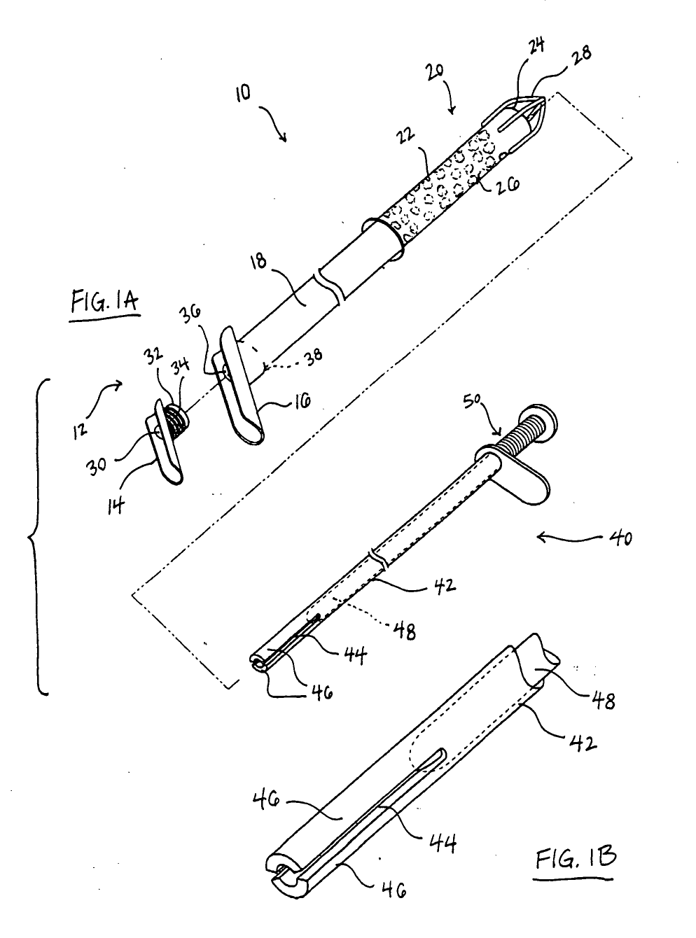

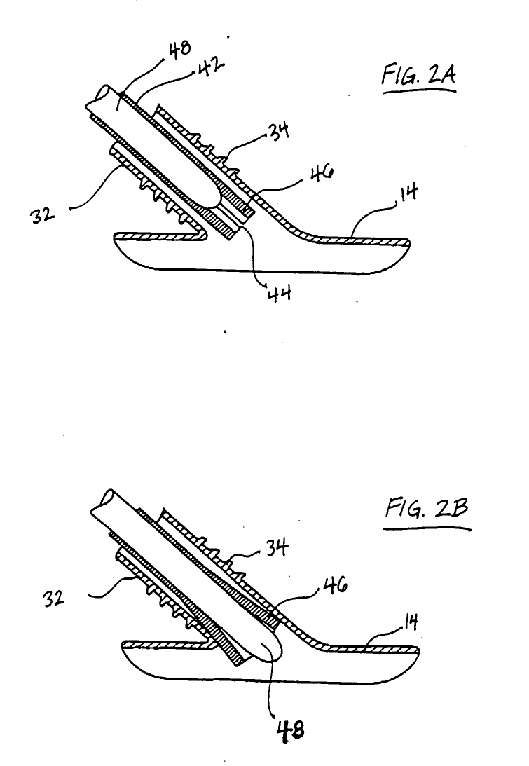

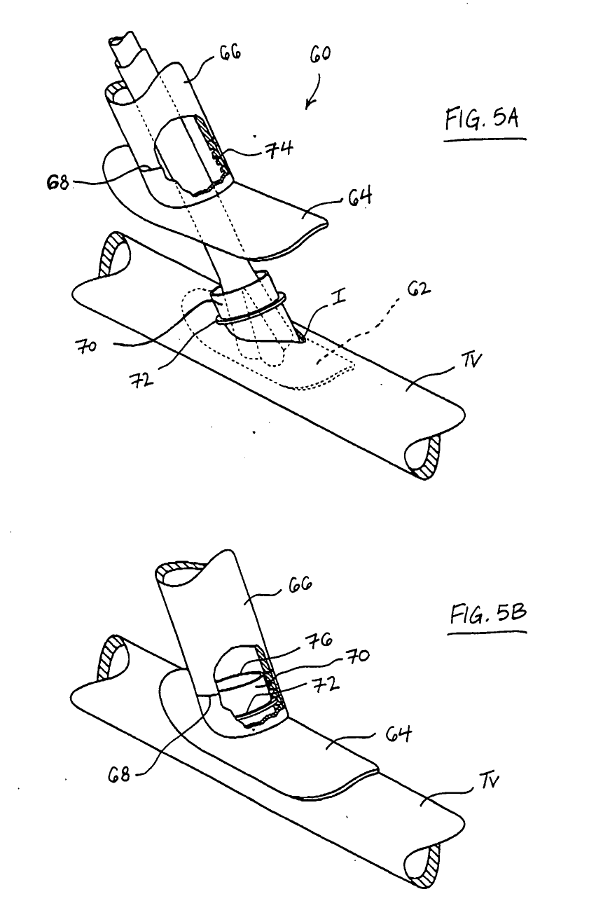

FIGS. 1A and 1B show a conduit 10 constructed according to one embodiment of the present invention. The conduit 10 is in the form of an elongated tubular body of vascular graft material, for example, autologous tissue, synthetic material, such as expanded PTFE, or a composite of tissue and synthetic material. One or more ends of the conduit 10 has an attachment portion 12 including a first securing component 14 and a second securing component 16. The conduit body may be formed integrally with one or both securing components (as exemplified by the embodiment of FIG. 3A). Alternatively, the conduit may be a separate element that is fixed to one or both securing components (as exemplified by the embodiment of FIG. 5A). If it is a separate element the conduit may be coupled to the securing component(s) via any suitable structure, for example, suture, fasteners, clamps, clips, expandable locking elements, etc. The same or similar coupling structure may be used to attach the body 18 of the conduit 10 to any structure that is disposed proximal to the attachment portion 12. In the illustrated and exemplary embodiment, the conduit 10 includes a device 20 for communicating with a heart chamber containing blood.

The device 20 comprises a tube 22 with an open end 24, optional openings 26 in the wall of the tube, and an optional cage 28 with struts for preventing blockage of the conduit 10. The device 20 is adapted to be positioned in the myocardium and is capable of withstanding myocardial contraction during systole so that the conduit 10 remains at least partially, and preferably completely, open during use. The device 20 may be constructed according to the teachings of co-pending, commonly-owned application Ser. No. 09/304,140, filed on May 3, 1999, and entitled “Methods and Devices for Placing a Conduit in Fluid Communication with a Target Vessel,” the entire subject matter of which is incorporated herein by reference.

The first and second securing components 14, 16 are coupled by a mechanism that applies sufficient force to maintain the two components in a desired relative position with respect to each other and a portion of the wall of a target vessel. The vessel wall is captured between the first and second securing components 14, 16 to secure the conduit to the target vessel. The first securing component 14 has a lumen 30 and an extension 32 with locking structure, such as ratchet teeth 34, threads, discrete rings, etc., for engaging the second securing component 16. As shown in FIG. 3A, the second securing component 16 has a lumen 36 that is generally aligned with the lumen 30 of the first securing component 14 (and the lumen of the conduit body 18). The second securing component 16 also has mating locking structure, such as grooves 38, for engaging the ratchet teeth 34 of the securing component 14.

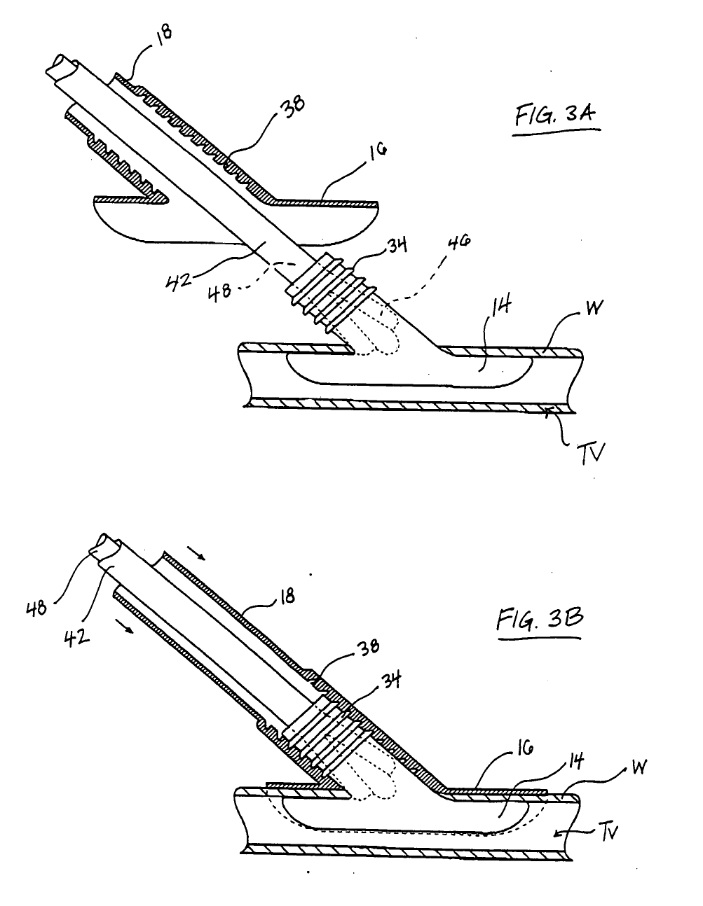

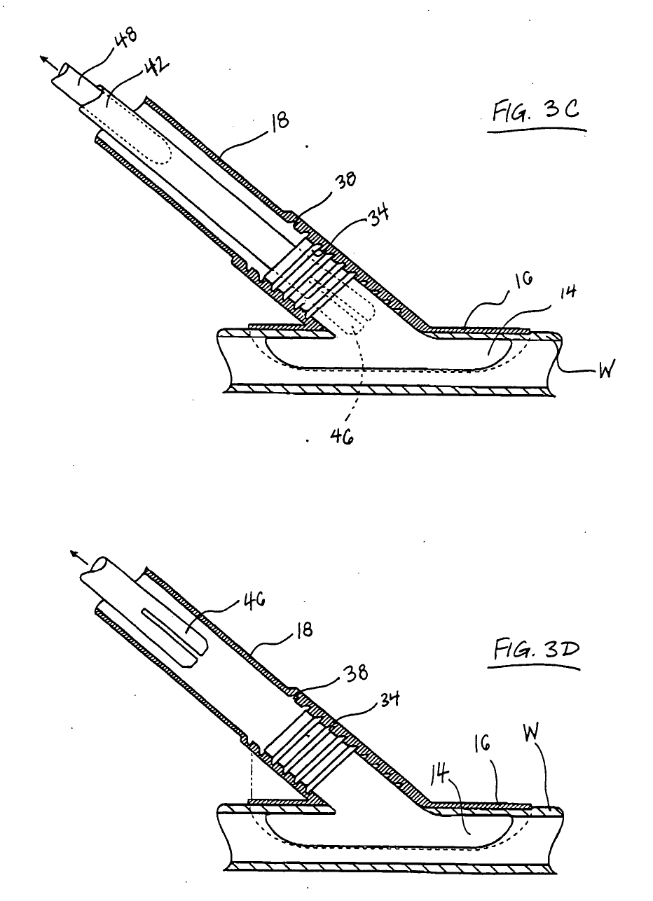

FIGS. 1A-1B and 2A-2B also show a delivery device 40 for use in deploying the conduit 10 (or a conduit constructed according to another embodiment of the invention). The illustrated delivery device 40 includes a sleeve 42 with a slit 44 at one end to form expandable arms 46, the arms being shown in a conduit-releasing position in FIGS. 1A and 1B. A shaft 48 is disposed in the sleeve 42 and is movable with respect thereto, for example, by a threaded attachment 50, as shown in FIG. 1B. Other means for imparting relative movement to the shaft 48 and sleeve 42 may be used instead, e.g., a bayonet coupling, lever assembly, friction fit, etc. The arms 46 of the delivery device 40 are placed in the lumen 30 of the first securing component 14 while the arms and the shaft 48 are in the conduit-releasing position, as shown in FIG. 2A. The device 10 is moved to the conduit-retaining position by sliding the shaft 48 distally (FIG. 2B). This forces the arms 46 of delivery device 40 to expand against the interior surface of the first securing component 14, and in particular the extension 32 of the securing component. When in this position the arms 46 of the sleeve 42 are positively engaged with the first securing component 14 and may be used to position the component 14 in the target vessel.

With reference to FIGS. 3A-3D, an exemplary method of securing a conduit to a target vessel will be described using the conduit 10 and the delivery device 40 for illustration. FIG. 3A shows the first securing component 14 retained by the delivery device 40, and positioned against the interior surface of the target vessel wall W. The delivery device 40 is supported in the position shown in FIG. 3A to retain the conduit. In this position, the device 40 contacts the luminal surface of the component 14, which may be undesirable given the fact this surface forms part of the blood flow path. The arms 46 of the device 40 thus may be coated with a substance or layer of suitable material, e.g., silicone, to prevent scratching or otherwise damaging the conduit's luminal surface which could adversely affect flow conditions during use.

Next, the second securing component 16 is moved along the sleeve 42 until the grooves 38 of component 16 engage the teeth 34 of the securing component 14. FIG. 3B shows the second securing component 16 after it has been moved against the exterior surface of the vessel wall W with the teeth 34 and grooves 38 locked in position. The vessel wall W is captured and compressed between the first and second securing components 14, 16 to secure the conduit 10 to the target vessel. The shaft 48 of delivery device 40 is then retracted, as shown in FIG. 3C, and the sleeve 42 is removed from the conduit 10, as shown in FIG. 3D. The result is a secure connection that provides hemostasis while leaving the majority of the target vessel lumen unoccluded adjacent the anastomosis site. The locking structure may be modified from that shown; for example, additional grooves 38 may be used to provide further adjustment for accommodating varying vessel wall thickness.

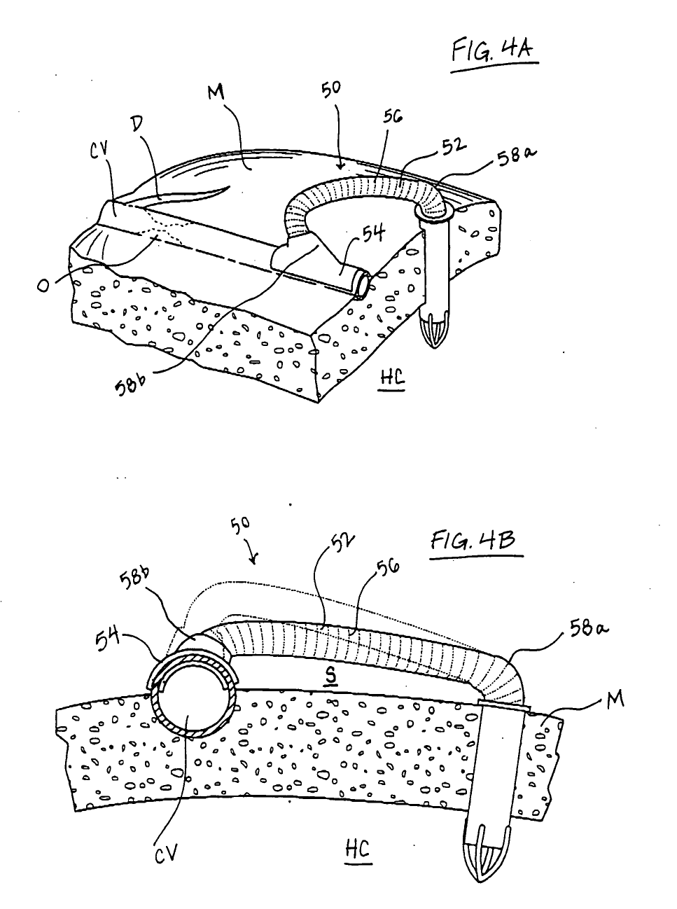

FIGS. 4A and 4B show a portion of a heart including a section of myocardium M, a coronary vessel CV, a side branch or diagonal vessel D, and an occlusion O which at least partially blocks blood flow from a native proximal source (to the left in FIG. 4A). A conduit 50 is positioned as described above to communicate a heart chamber HC with the coronary vessel CV. The conduit 50 has an alternative construction and is configured to assume a desired profile or orientation with respect to the myocardium when deployed. The conduit 50 has a body 52 with a lumen substantially free of discontinuities, and first and second securing components, one of which is visible at 54 in FIG. 4A, and a transmyocardial device in communication with the heart chamber HC. The conduit body 52 is provided with a reinforcing component 56 that prevents the conduit from kinking and may also aid in maintaining the conduit in the desired, preselected orientation. The reinforcing component 56 may take any suitable form, for example, a nickel titanium coil, elongate struts, a polymeric or metallic skeleton or frame that may be configured similarly to a stent disposed along all or a portion of the conduit, a reinforcing layer or laminate, etc.

The conduit 50 of this embodiment is formed to assume a low profile orientation when in an unbiased state that minimizes the space S between the myocardial tissue and the conduit body 52 (FIG. 4B). This may reduce the likelihood of the conduit being crushed or kinked, for example, by the patient's chest wall, during or subsequent to completion of the procedure. Additionally, the conduit 50 is constructed to assume an orientation having a component that lies toward the axis of the target vessel, which may be desirable for flow dynamics. It should be noted that the conduit 50 of this embodiment includes two preferred features of the invention, namely, a substantially continuous inner lumen free of discontinuities and a preselected orientation when deployed. It will be appreciated, though, that the invention may be practiced utilizing these (and other) features either alone or in combination.

The conduit 50 may be formed to assume a specific orientation by providing the entire conduit body 52 with a shape memory component, such as a nickel-titanium alloy coil; or, alternatively, one or more sections of the conduit 50 may be provided with specifically shaped structure. The illustrated conduit 50 is provided with guide portions 58a, 58b for orienting the conduit portion in the preselected orientation described above. One of these portions 58a is located adjacent the end of the conduit 50 that is placed in communication with the heart chamber HC and preferably directs the conduit body 52 to a generally parallel orientation with respect to the myocardium M. Another of these portions 58b is located adjacent the end of the conduit 50 provided with the first and second securing components and, in the illustrated construction, is secured to the second securing component 54 so as to extend away at an angle, e.g., approximately 45°. The portion 58b also preferably directs the conduit body 52 to a generally parallel orientation and along with the portion 58a orients the conduit 50 in the low profile position of FIG. 4B (an alternative, higher profile position being shown in phantom).

The conduit 50 may be flexible to allow it to be biased from the preselected orientation, for example, during deployment of the conduit; the conduit would then return to its preselected orientation after attachment to the blood source and the target vessel. It should be recognized that the conduits of the invention may be constructed to assume various preselected orientations that will depend, at least in part, on the application in which they are used. As an example, in some procedures, for instance, treating peripheral vascular disease or forming an arteriovenous shunt, it may be desirable that the conduit assume a specific orientation in order to be better accommodated by adjacent anatomical structure. In other procedures, it may be desirable to have the conduit follow a short path between the blood source and the target vessel, for example, to minimize the amount of autologous vessel used for each bypass. As a result, this aspect of the invention is not limited to any particular preselected conduit orientations or any specific means of achieving such orientations.

FIGS. 5A and 5B show another embodiment of the invention comprising a conduit 60 which includes a first securing component 62, a second securing component 64, and a conduit body 66. The conduit body 66 is a separate tubular member secured to the second securing component 64 at a junction 68. The first securing component 62 has a stem 70 adapted to extend at least partially through an incision I formed in the target vessel TV. The stem 70 carries one or more locking elements 72 designed to mate with one or more locking grooves 74 formed on the interior of the second securing component 64 and/or the conduit body 66 (FIG. 5B). The conduit body 66 extends away from the second securing component 64 at a desired angle, e.g., 45°, so that the conduit assumes a desired orientation with respect to the target vessel. It will be recognized, however, that the conduit could extend away at a different angle, for example, 30°, 60° or 90°.

Another feature of the conduit 60, as well as the conduits 10 and 50 described above, is that—except for the incision through which the first securing component is passed—the connection or anastomosis is made without penetrating the target vessel wall. The target vessel wall is held between the securing components to place the conduit in fluid communication with target vessel lumen. This feature of the invention contrasts with prior art anastomotic couplers that include one or more elements that pass through or substantially penetrate the vessel wall. The invention may be practiced with one or more portions to slightly pierce, but not significantly penetrate, the tissue. As above, this feature of the invention may be used independently of the other coupling features disclosed herein.

The force applying mechanism of the conduit 10 comprises teeth 34 and grooves 38 which interlock to fix the relative position of the first and second securing components 14, 16 (or, alternatively, substantially fix their position so as to permit a limited amount of relative movement between the components). The conduit 10 (as well as the other conduits disclosed herein, such as conduits 50 and 60) may be used with suitable alternative force-applying mechanisms, for example, any of those disclosed in co-pending, commonly-owned application Ser. No. 09/393,130, filed on Sep. 10, 1999 and entitled “Anastomotic Methods and Devices for Placing a Target Vessel in Fluid Communication with a Source of Blood,” the entire subject matter of which is incorporated herein by reference.

Similarly, the first and second securing components of the conduit attachment portion may be formed of any suitable material, such as those materials explicitly listed herein or described in the applications incorporated by reference herein. Additionally, the securing components may be coated or impregnated with various desired materials, including any of these materials. Suitable exemplary materials include titanium, nickel-titanium alloy, stainless steel, expanded polytetrafluoroethylene (ePTFE), polyurethane, polyamides, polyimides, fluoroethylpolypropylene (FEP) and polypropylfluorinated amines (PFA), silicones, etc. In sum, the invention may be used with any suitable blood-compatible materials.

The conduit 60, and in particular the conduit body 66, of this embodiment has a lumen defined by the inner surface of the conduit body 66 and the inner surface of the stem 70 of the first securing component 62. The lumen that forms the blood flow path is therefore not completely free of discontinuities because the end 76 of the stem 70 of securing component 62 forms a step (FIG. 5B). This is also true for the conduit 10, as shown best in FIG. 3D. Some embodiments of the invention, however, for example, as described below, include a lumen that is free (or substantially free) of discontinuities to promote continuous blood flow that is more laminar than turbulent in nature.

As mentioned above, the first and second conduit securing components may be coupled or biased toward each other by one or more lengths of suture, wire, or wire-like material. FIGS. 6A-6B show a conduit 80 including a first securing component 82, a second securing component 84, a conduit body 86, and a mechanism 88, for biasing the securing components toward each other. FIG. 6A shows the securing components 82, 84 in their tissue-releasing position, with the first securing component 82 placed inside the lumen of the target vessel TV through an incision I. The mechanism 88 includes several elongate elements 90, such as lengths of suture, that are coupled to the first and second securing components 82, 84 by extending through openings 92 therein. The elements 90 preferably pass through the incision I and make the anastomosis without substantially piercing or penetrating the target vessel wall.

A suitable device, such as the forceps-type instrument 94 with shaped tips 96, may be used to hold and manipulate the conduit 80 while delivering the first securing component 82 through the incision I and into the target vessel lumen. This type of device may be used if it is desired not to engage the blood-contacting surface(s), although any suitable delivery tool may be used. The second securing component 84 is then slid over the elements 90 into its tissue-capturing position, as shown in FIG. 6B. The elements 90 are tied off and trimmed to produce a fluid tight anastomosis.

It will be noted from FIG. 6B that the conduit 80, and specifically conduit body 86, is preformed to assume a preselected orientation when in an unbiased state. FIG. 6A shows the conduit 80 biased to a different orientation, for example, by another pair of forceps (not shown), while FIG. 6B shows the conduit 80 after the biasing force has been removed. The illustrated and exemplary configuration is curved to assume a low profile relative to the target vessel TV. The conduit 80 may be preformed in various ways. For instance, the conduit body 86 may carry a coil 98 to bias the conduit 80 to the orientation of FIG. 6B.

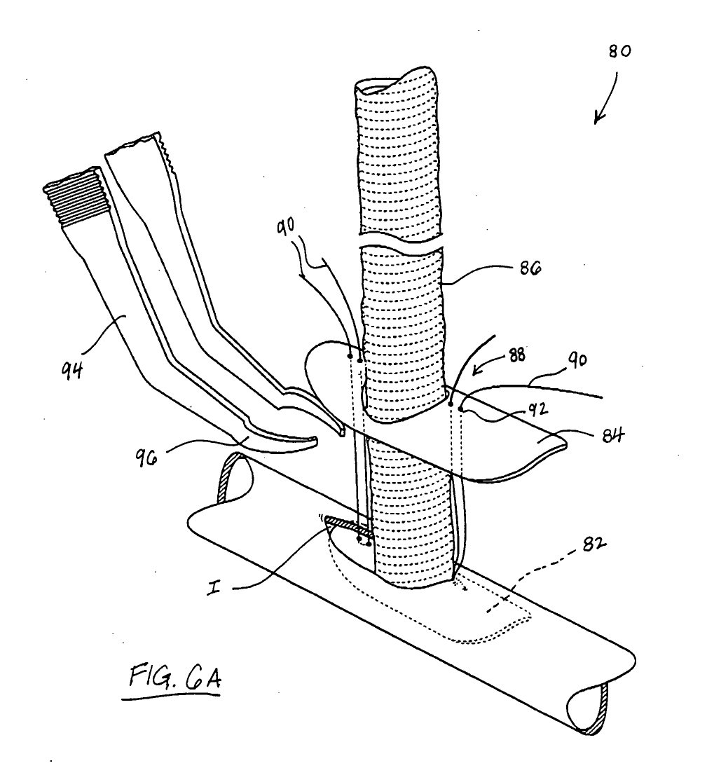

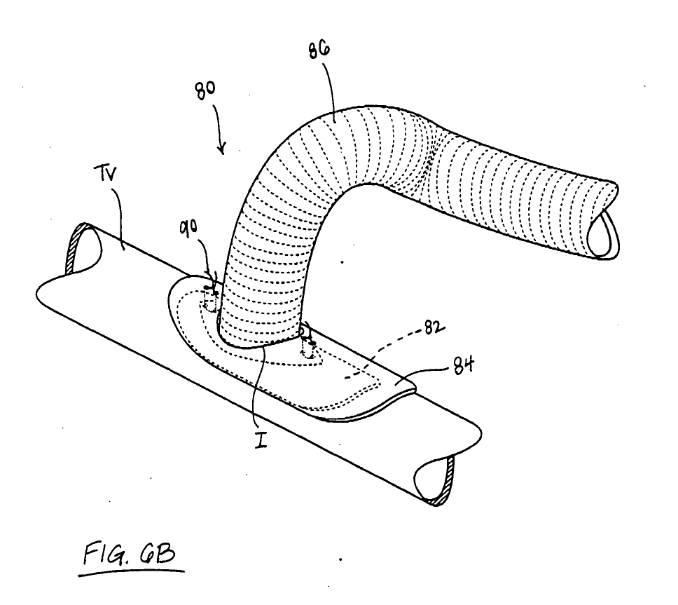

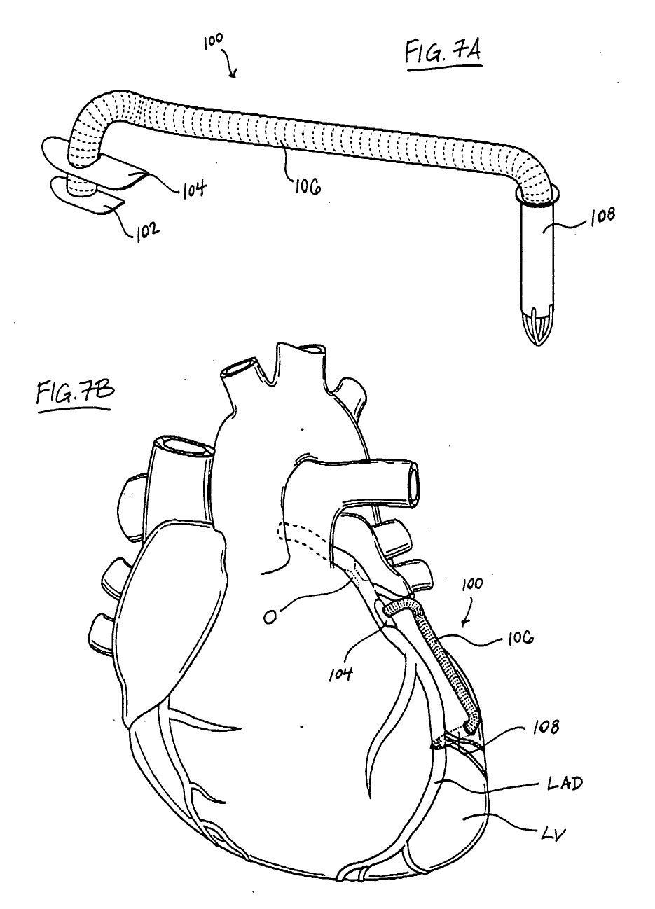

FIGS. 7A-7B show a conduit 100, similar to conduit 80 in that it is preformed to assume a desired orientation in use, including first and second securing components 102, 104, conduit body 106, and a device 108 adapted to be placed in fluid communication with a heart chamber containing blood. The first and second conduit securing components 102, 104 may be biased to and held in a tissue-capturing position by any of the mechanisms disclosed or incorporated by reference herein. FIG. 7B illustrates one application of the invention wherein the conduit 100 forms a ventricular bypass graft. The first and second securing components 102, 104 are secured to the target vessel (the LAD in this embodiment) at a site distal to an occlusion O that blocks or impedes blood flow to the distal vascular bed. The device 108 is placed in the myocardium with its open end communicating with the left ventricle LV, and the conduit body 106 delivers blood from the ventricle LV to the LAD to perfuse the myocardium distal to the occlusion O. The conduit body 106, which can comprise tissue or synthetic material, may be everted over the end of the device 108 if desired.

The conduit body may be either separate from or integrally formed with the securing component to which it is coupled. The conduits of the embodiments illustrated in FIGS. 5A-5B, 6A-6B and 7A-7B include conduit bodies that are separate from and fixed to one of the securing components. The conduit body includes a reinforcing component that is attached to one of the securing components in a suitable manner, e.g., by adhesive bonding, welding, brazing, fasteners, etc., thereby securing the conduit to the securing component. In contrast, the conduits of the embodiments shown in FIGS. 1A-1B, 2A-2B and 3A-3D include conduit bodies that are integrally formed with one of the securing components in a suitable manner, e.g., a molding or extrusion process. Finally, FIGS. 4A-4B show an embodiment that is generic as to the construction of the conduit body and conduit securing components.

Further, it will noted that in the embodiments of FIGS. 6A-6B and 7A-7B the conduit body (86, 106) is joined to the first securing component (82, 102), whereas in the embodiments of FIGS. 1A-1B, 2A-2B, 3A-3D and 5A-5B the conduit body (18, 66) is joined to the second securing component (16, 64). In either case the conduit body may be integrally formed with the securing component. In sum, the conduits of the invention may be coupled to either securing component, and they may comprise a separate or integrally formed part of the securing component.

FIGS. 8-11B show one possible construction to join a separate conduit body and a conduit securing component. A conduit 110 includes first and second securing components 112, 114, conduit body 116, and a reinforcing component 118. A suitable device (such as instrument 94) may be used to manipulate the conduit 110 and deliver the first securing component 112 into a target vessel lumen (not shown). Securing means 120 extends through complimentarily formed openings 122 in the securing components 112, 114 and are used to retain the securing components in their tissue-capturing position, as explained above. The conduit body 116 is secured to the first securing component 112 at a junction 124 (FIG. 8), which is preferably a flexible connection that allows the conduit to be manipulated and bent during use. The conduit 110 may be formed to assume a desired orientation when unbiased, for example, as described above. Alternatively or additionally, the conduit 110 may be substantially resilient or floppy at the junction 124 (and the conduit body 116).

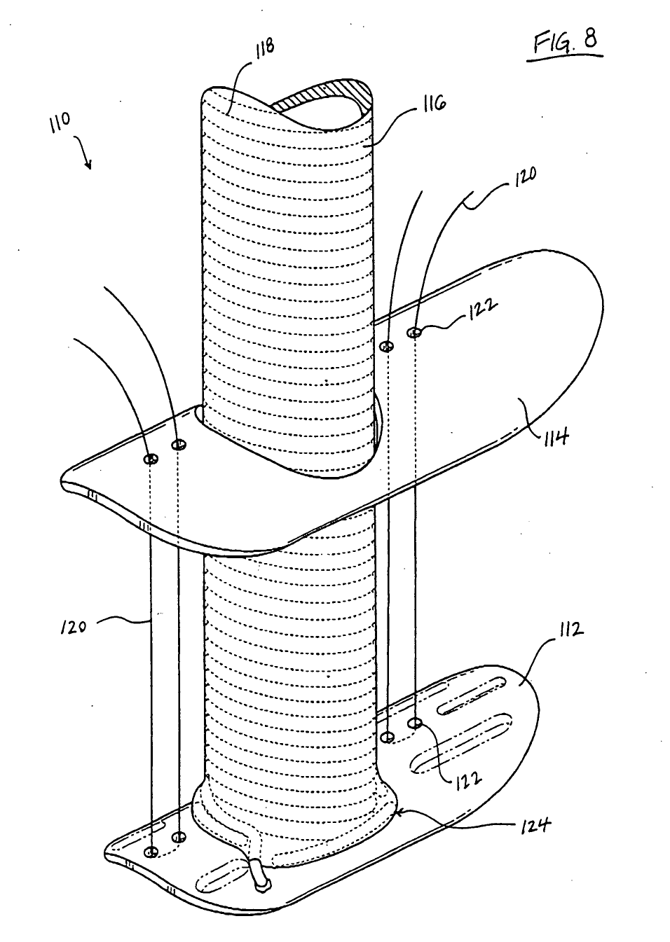

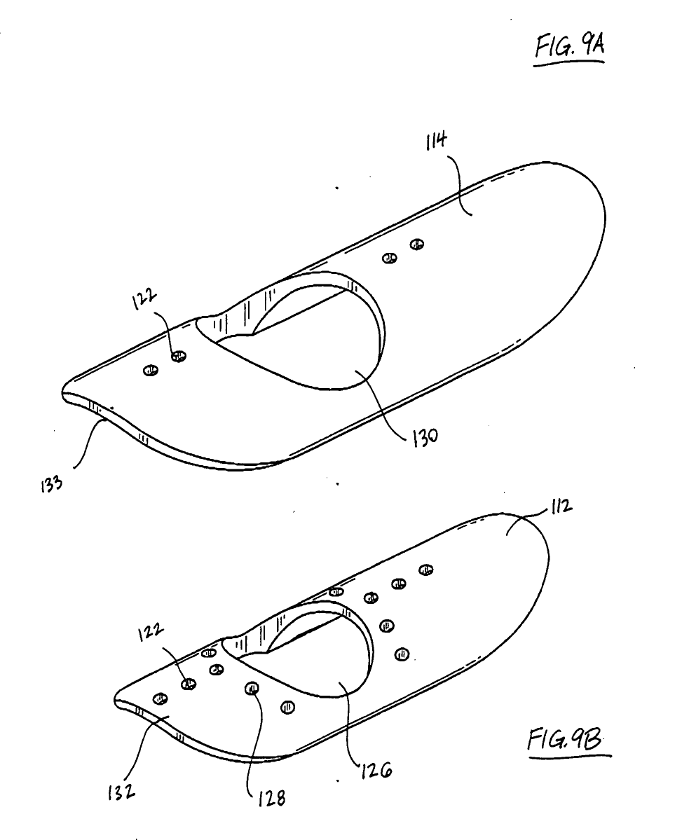

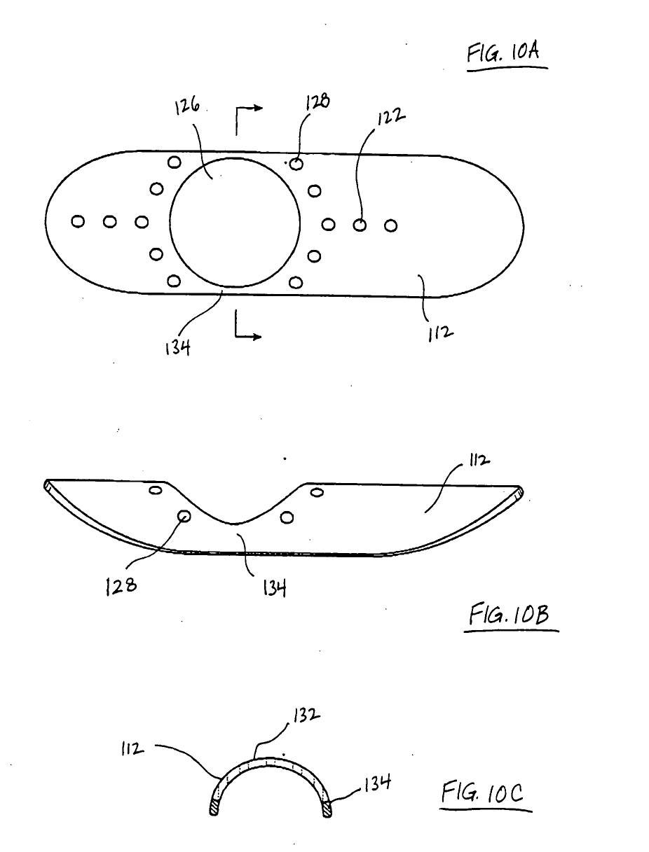

FIGS. 9A-9B and 10A-10C are enlarged views of the first and second securing components 112, 114, which are preferably configured to substantially mate when moved to their tissue-capturing position on opposite surfaces of the target vessel wall. The first securing component 112 has a central opening 126 which communicates with the open end of the conduit body 116 when attached thereto. A plurality of apertures 128 are formed in the first securing component 112 and are used to secure the reinforcing component 118 to the component 112. The second securing component 114 has a central opening 130 sized and configured to receive the conduit body 116 and allow the securing components 114, 116 to be moved toward each other in use.

The shape of the conduit securing components may of course be varied from those shown. The arcuate configurations shown in the Figures are preferred because the first securing component 112 has a curved surface 132 which contacts and supports the interior surface of the target vessel wall while occupying a minimal amount of the vessel lumen. FIG. 10C shows the preferred, arcuate cross-sectional shape of the first securing component 112 including reduced size portions 134 located on each side of the central opening 130. The wall 134 of the securing component 112 extends over an angle which, in the illustrated embodiment, is approximately 180°. The angle may of course be different from that shown. As an example, the angle is preferably within a range of from about 45° to 330°, more preferably about 90° to 300°, and most preferably about 120° to 270°. Nonetheless, it should be recognized that the first securing component of the invention can include an intraluminal portion that extends substantially or completely 360° around the target vessel circumference.

The angular configuration of the securing component may also vary along its length (generally along the axis of the target vessel), as well as along its width (generally along the circumference of the target vessel wall). The shape of surface 133 of second securing component 114 (FIG. 9A) preferably, but not necessarily, substantially matches that of the first securing component surface 132 to provide a tighter seal against the tissue captured between the components.

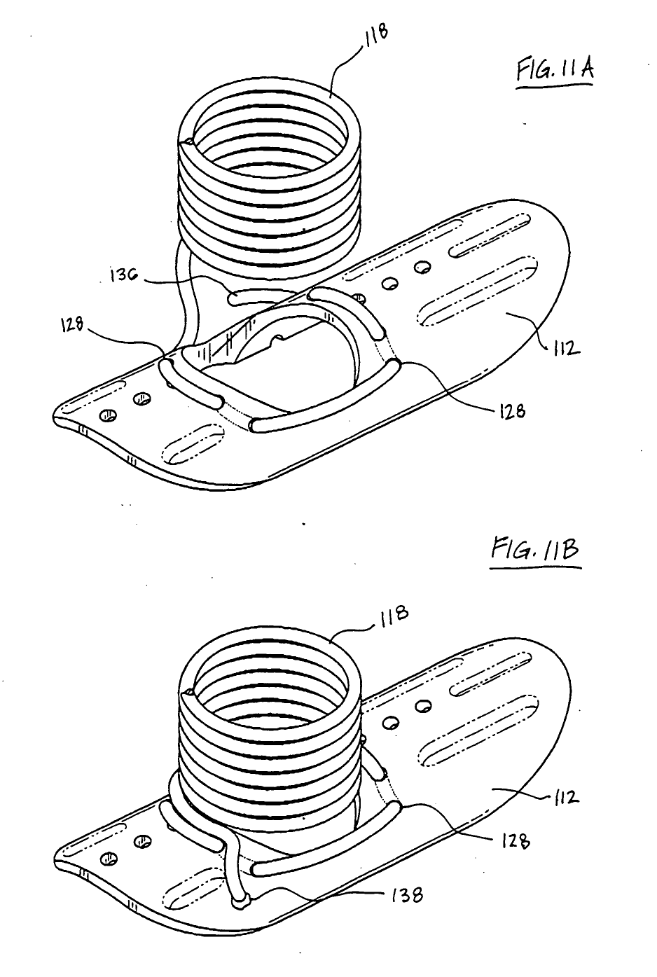

FIGS. 11A-11B illustrate in detail an exemplary manner of attaching the conduit 110 to the first securing component 112, the component 112 including a skeleton frame in this embodiment. The material forming the walls of the conduit body 116 is omitted for clarity, which leaves only the reinforcing component 118. The conduit reinforcing component 118 is preferably in the form of a thin wire, e.g., a stainless steel or nickel-titanium alloy coil, having an end 136 which is threaded through the apertures 128 (FIG. 11A) and then fixed at 138 to the first securing component 112, for example, by a spot weld (FIG. 11B). This orients the conduit 110 with respect to the first securing component 112 with the conduit body 116 fluidly sealed adjacent the central opening 126 of the component.

Additionally, because the conduit body 116 in this embodiment is only attached to the first securing component 112 by one or a few turns of the wire forming the reinforcing component 118, the result is a flexible connection that permits the conduit body to be easily moved relative to the securing component. A benefit of this feature is that the conduit body may be manipulated and moved in various directions and to various degrees with respect to the securing components. This allows manipulation during use, for example, to deliver the conduit in a minimally invasive manner. Another benefit of this feature is that the first and second securing components 112, 114 may be secured to the target vessel wall and the remaining portion of the conduit 110 then manipulated without transmitting excessive force to the target vessel due to the flexible connection. It will be appreciated that threading or tying the reinforcing component to the conduit securing component is only one possible means for affixing these members. Other suitable means include brazing or welding, adhesive bonding, crimping or fastening.

FIG. 12 shows a conduit 140 constructed according to another embodiment of the invention in order to provide better visualization of the working end of the device, and in particular a first securing component 142, during delivery in to the target vessel TV. The conduit 140 has essentially the same construction as the conduit 80 described above in connection with FIGS. 6A-6B except the second securing component 144 is angled with respect to the securing component 142 (at 90° in the illustrated embodiment). This allows easy visualization of the incision I in the TV while delivering the leading end of the first securing component 142 into the vessel lumen. After placement and proper positioning of the first securing component 142 the second securing component 144 is moved down and secured, e.g., by sutures 146. An alternative embodiment to provide enhanced visualization utilizes a second securing component formed of a transparent or translucent material that allows the user to view the first securing component during deployment. Another embodiment uses a visual or auditory indicator to show that one or both components has reached a desired position.

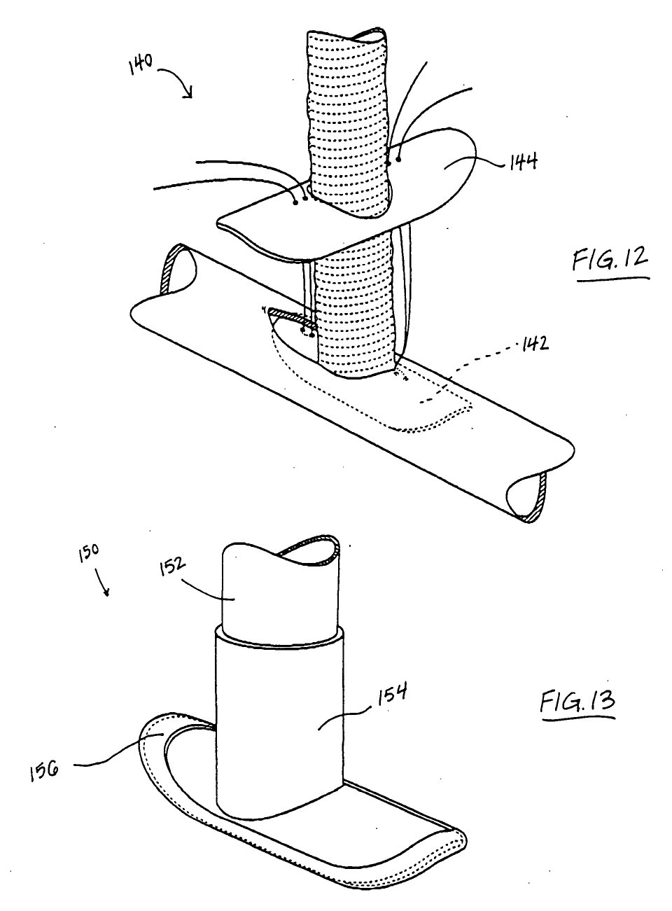

FIG. 13 shows a conduit component 150 constructed according to yet another embodiment of the invention. The component 150 preferably comprises the conduit member that is in blood contact and to that end includes a liner 152 formed of a material possessing beneficial blood interface properties, such as ePTFE, Dacron®, or another synthetic vascular graft material. The liner 152 is placed within a conduit securing component 154 and an end 156 of the liner is preferably everted, for example, over an end of the vessel wall-contacting portion of the securing component 154. The liner 152 alone may form the conduit body or it may do so in conjunction with an autologous (or other tissue) vessel. As still another alternative an autologous vessel alone may be used. The liner 152 may be attached to the conduit securing component by any suitable means, e.g., adhesives or suture.

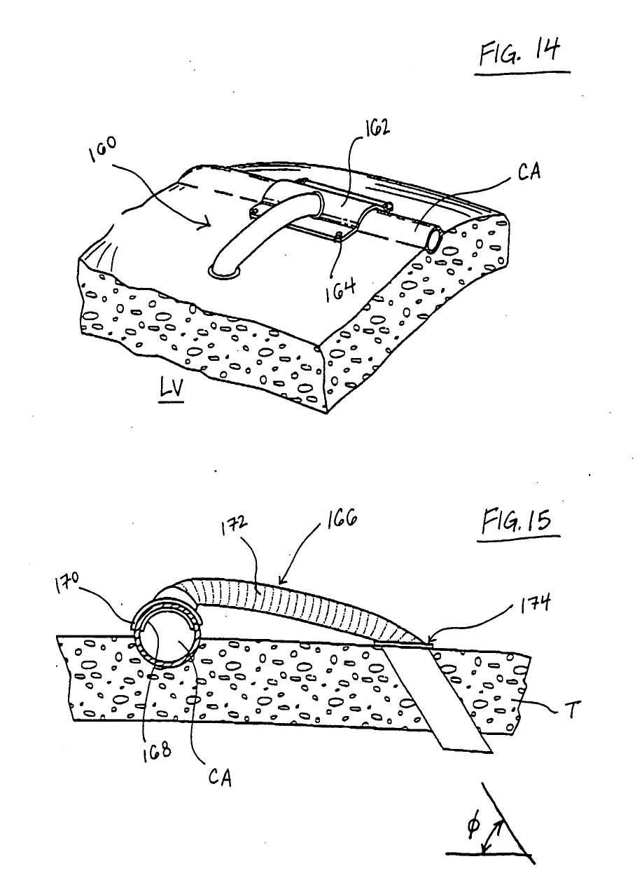

FIG. 14 shows a conduit 160, which is constructed according to another embodiment of the invention and placed in communication with a coronary artery CA and the left ventricle LV. The conduit 160 enters the side of the artery CA rather than its top, which produces a flatter configuration, and secured by a securing component 162 overlying the artery. The securing component 162 may be held by sutures 164 or another suitable attachment means, e.g., the engagement mechanisms described above.

FIG. 15 shows a conduit 166 with first and second securing components 168, 170, a conduit body 172, and an inlet portion 174 located in myocardial tissue T. Rather than passing perpendicularly through the myocardial tissue T, the inlet portion 174 extends at an angle φ which may be, for example, in the range of about 30° to about 90°. The conduit body 172 is able to extend toward the artery CA along a flatter line to reduce torque on the vessel and conduit and prevent kinking of conduit 166.

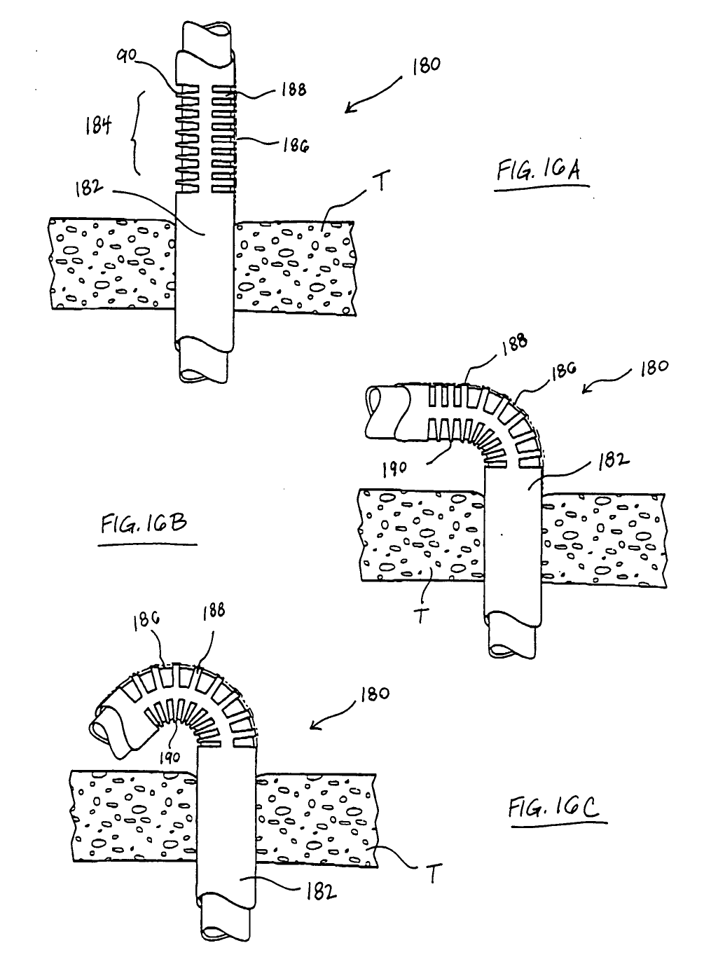

FIGS. 16A-16C show another embodiment of the invention which provides a conduit that may be adjustably positioned with respect to the heart wall and retained in place. A conduit 180 is positioned in myocardial tissue T and preferably has a solid or substantially solid wall portion 182 and an adjustable portion 184. The adjustable conduit portion 184 is articulated to allow the relative position of the inlet and outlet ends of the conduit 180 to be changed within a wide range of adjustability. The illustrated structure for facilitating articulation of the conduit portion 184 comprises ring-shaped cuts 186 which define ring-shaped bands 188. The cuts 186 allow the conduit portion 184 to partially collapse in order to change the conduit position. The cuts 186 may be tapered by removing more material, and thus form a narrower area 190 of each band 188.

FIG. 16B shows the conduit 180 moved from the position shown in FIG. 13A to an approximately 90° position relative the myocardial tissue T. The thin band areas 190 allow the inside of the conduit 180 to collapse without adjacent bands 188 on that side of the conduit abutting. FIG. 16C shows the conduit 180 moved to another alternative position wherein the axes of the inlet and outlet ends of the conduit form an acute angle, approximately 45° in the Figure. It will be recognized that other suitable conduit structures may be used to achieve the same or more adjustability that is provided by this embodiment of the invention. For example, rather than using a metal hypo tube that is laser cut to allow collapsing as shown in FIGS. 16A-16C, an articulated conduit could comprise the thin band areas 190 on both sides of the conduit 180 to allow bi-directional bending, or a dual-coil design to allow bending in multiple directions. It will similarly be appreciated that the conduit of the invention may move over a desired range(s) of angles, for instance, preferably within about 180°, and more preferably within about 150°.

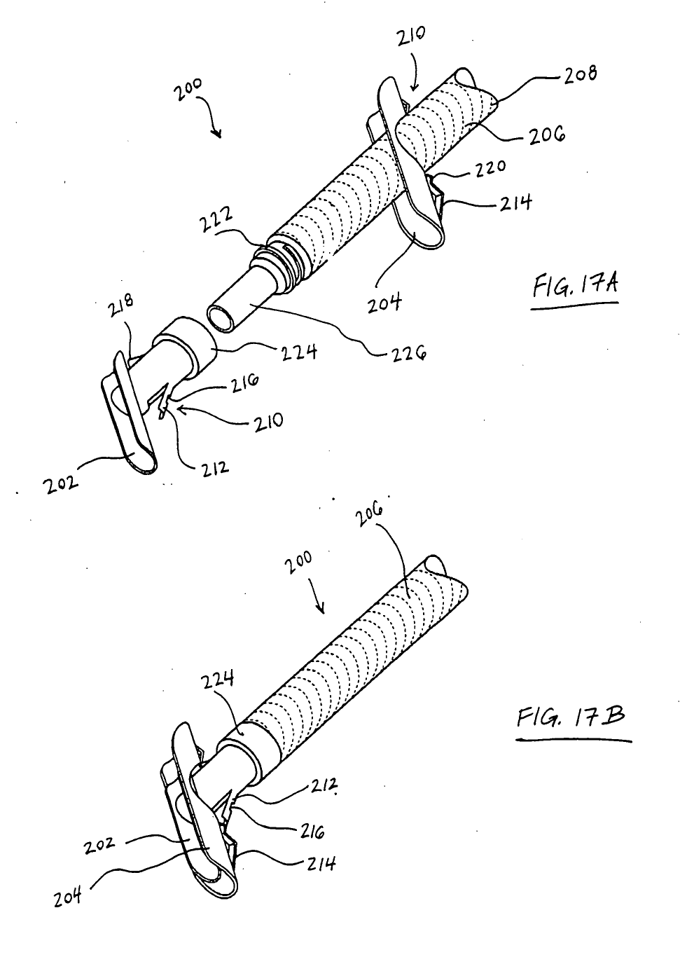

FIGS. 17A-17B show a conduit constructed according to another embodiment of the invention. The conduit is indicated generally at 200 includes first and second securing components 202, 204, conduit body 206, and a reinforcing component 208. A suitable device (such as the instruments described above) may be used to manipulate the conduit and deliver the first securing component 202 into a target vessel lumen. A coupling mechanism 210 is used to fix the relative position of the first and second securing components 202, 204 and, as shown in FIG. 17A, comprises mating tabs 212, 214. The tabs 212 are carried by the first securing component 202 while the tabs 214 are carried by the second securing component 204. The tabs 212, 214, which are preferably thin, leaf spring-like elements but take other configurations, interlock to maintain the conduit securing components 202, 204 in their tissue-capturing position (shown in FIG. 17B).

The tabs 212, 214 (or another locking means, such as a fastener integral with the securing components or a separate coupling element that engages the securing components) may include means for providing an audio or visual indication to the user that the securing components are in their correct, tissue capturing position. The tabs 212, 214 click into place when in the desired position, and they may lock in a single position only or in one of several positions to provide adjustability, for example, to accommodate different vessel wall sizes or amounts of tissue. The coupling mechanism 210 is of course only one example of a coupling for use with the invention.

In the illustrated embodiment, the tabs 212 have openings 216 and ends 218, while the tabs 214 have ends 220. During engagement the tabs 214 slide along the tabs 212 until the ends 220 of tabs 214 drop into the openings 216 of tabs 212. This corresponds to a first position (not shown) of the coupling mechanism 210. From here the tabs 214 may be slid further until their ends 220 drop under the ends 218 of the tabs 212. This corresponds to a second position (shown in FIG. 17B) of the coupling mechanism 210. The ends 220 of tabs 214 push against the tabs 212 to produce a resultant force that biases the first and second securing components 202, 204 toward each other, thereby enhancing the attachment to and seal with the target vessel.

The conduit 200 shown in FIGS. 17A-17B includes a conduit body 206 that is formed as a separate component coupled to at least one of the securing components. The conduit body 206 has an attachment portion, such as quick-coupling threads 222, adapted to engage an attachment portion carried by the first securing component 202, for example, mating threads located inside a collar 224 of the securing component 202. The attachment portions may be configured to attach the conduit to the securing component in either a fixed or removable manner, and are designed to effect a fluid-tight coupling. The conduit 200 preferably comprises a length of tubing 226 joined to the conduit body 206 and extending through the first securing component 202 to provide a continuous lumen that defines a blood flow path substantially free of discontinuities.

FIGS. 18A-18B show two conduit securing components constructed according to other embodiments of the invention. The securing component 220 of FIG. 18A has a saddle-shaped body configured to conform to the shape of a vessel wall and a stem 224 defining a lumen 226. The lumen 226 may form part of the blood flow path or it may receive a tubular member (not shown) that defines the blood flow path. The stem 224 is fixed to the body by a rib 228 to orient the blood flow path in a desired direction. The size, shape, rigidity, or other characteristics of the rib 228 may be altered to achieve a component having the desired configuration and flexibility.

FIG. 18B shows a securing component 230 with a saddle-shaped body 232 and a stem 234 defining a lumen 236, as described above. The securing component 230 has one or more stabilizers 238 for contacting tissue and maintaining the relative position of the blood flow path with respect thereto. The stabilizers 138 may be rigid, flexible, malleable, etc. The conduit body 232 includes an internal support in the form of a frame or skeleton 240 to provide a desired degree of stiffness or flexibility, or to allow the component 230 to be custom fit to a target vessel. An exemplary construction uses a frame 240 of nickel-titanium alloy coated with silicone.

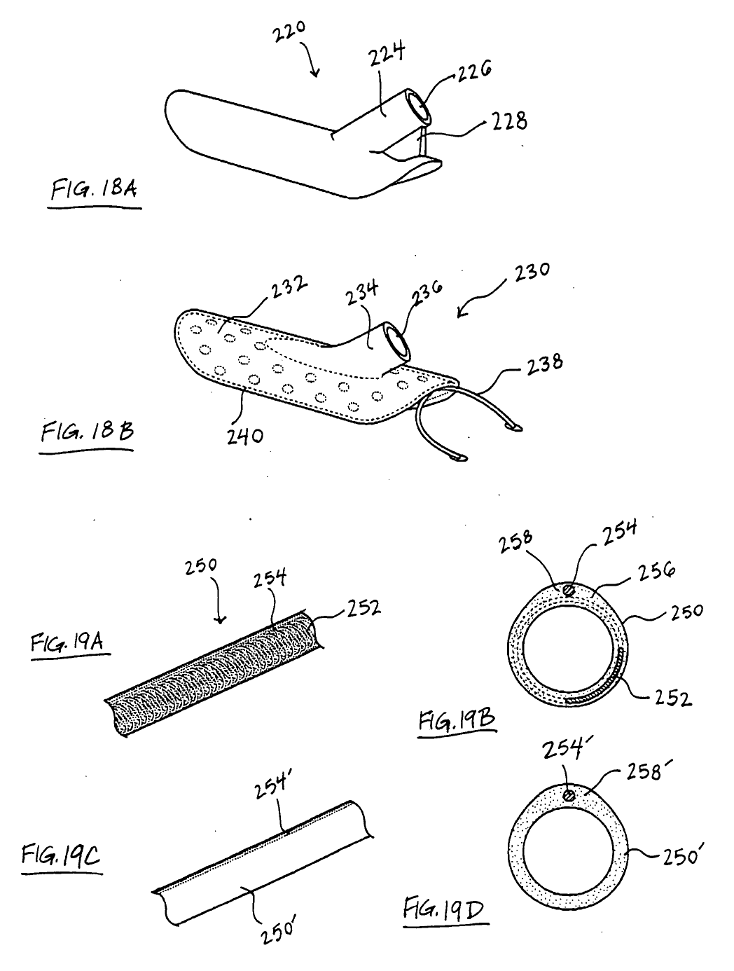

FIGS. 19A-19B show a conduit body 250 constructed according to another embodiment of the invention and including a reinforcing coil 252 and a reinforcing rail 254 encased in a suitable conduit material 256. The coil 252 and rail 254 are sized, configured and formed of a material, e.g., round wire, that allows the conduit body 250 to maintain its orientation after being bent to any of various positions and released. As shown in FIG. 19B, the rail 254 may be encased in a raised section 258 of the conduit body 250. The rail 254 and coil 252 may be used together (as exemplified by the conduit 250 of FIGS. 19A-19B), or they may be used together (as exemplified by the conduit 250′ of FIGS. 19C-19D).

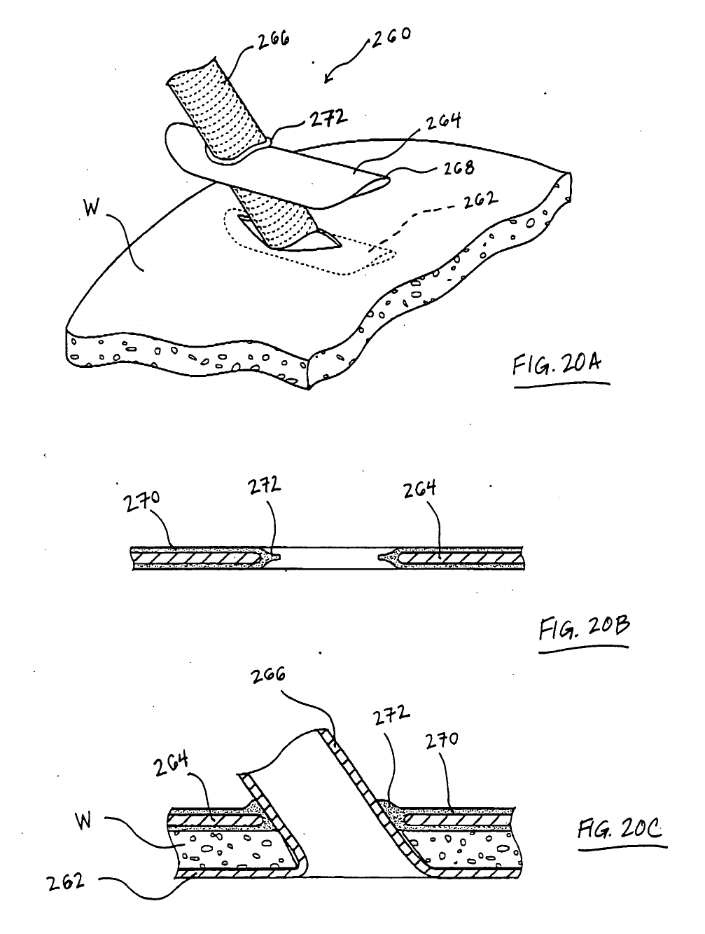

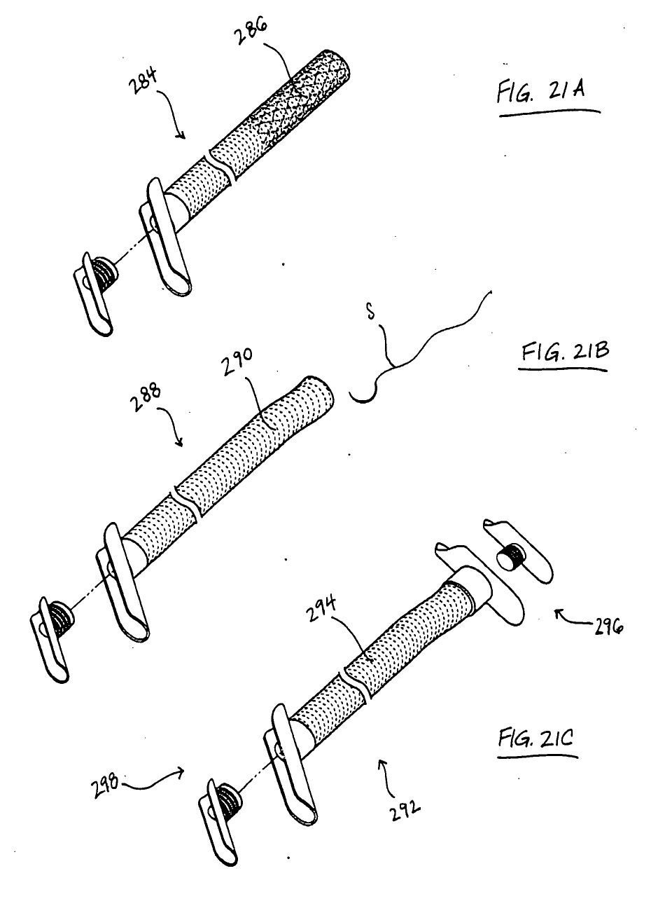

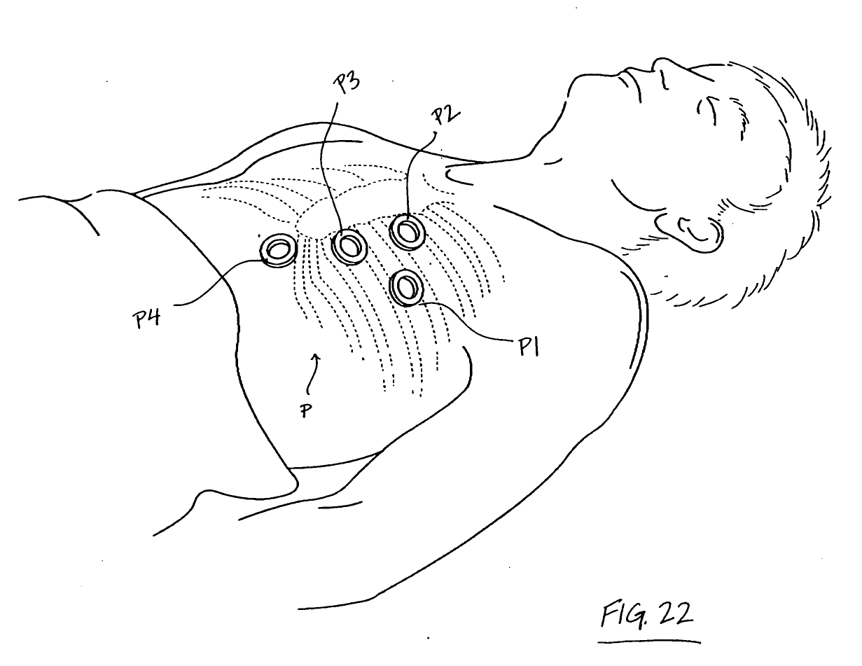

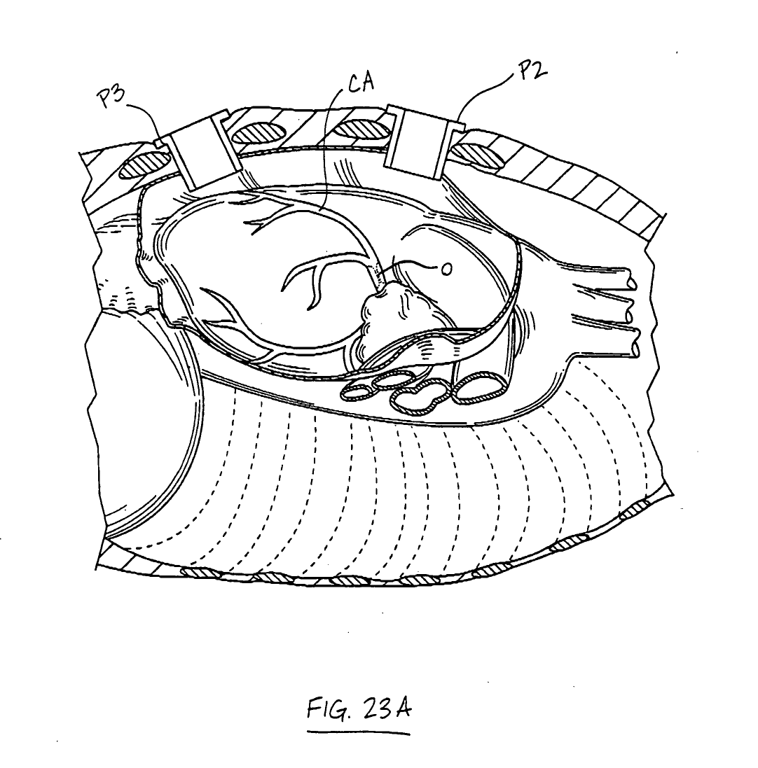

FIGS. 20A-20C show a conduit 260 constructed according to another embodiment of the invention. The conduit 260 includes first and second securing components 262, 264 and a conduit body 266. The second securing component 264 includes an arcuate body 268 with an opening that receives the conduit body 266. The arcuate body 268 includes a seal 270 with a ring-shaped portion 272 disposed around all or part of the opening that receives the conduit body 266 (FIG. 20B). After placing the first securing component 262 through an incision in a vessel wall W the second securing component 264 is slid down to engage the tissue, as explained above.

The seal 270 may be, for example, a silicone coating applied to the arcuate body 268 which slides along the exterior of the conduit body 266 during deployment. Once the conduit 260 has been secured to the wall W of the target vessel, the ring-shaped portion 272 of the seal 270 acts as a gasket to provide hemostasis at the incision (FIG. 20C). While the seal 270 is shown disposed over the entire second securing component 264, it could instead comprise a ring located adjacent the opening in the arcuate body 268 of second securing component 264.

It will be understood that many aspects of the invention may be practiced irrespective of the particular source of blood or the specific manner in which the conduit is secured to the either the blood source or the target vessel. FIGS. 21A-21C show exemplary conduits constructed according to the invention which utilize alternative means for securing conduit in fluid communication with a hollow body. Each conduit includes an attachment portion 280 for securement to a target vessel and a conduit body 282.

FIG. 21A shows a conduit 284 with a stent 286 for attaching the conduit to a source of blood (not shown). The stent 286 may be formed of any material and may be self-expanding or pressure-expandable. FIG. 21B shows a conduit 288 in which the conduit body 290 is not provided with a coupling mechanism for attachment to a source of blood. Rather, the conduit body 290 is simply sutured by conventional needle and suture S to the tissue at the blood source, for example, the wall of another vessel. FIG. 21C shows still another conduit 292 wherein the conduit body 294 is provided with a second attachment portion 296 constructed the same as or similar to the portion 298. It will be recognized that FIGS. 21A-21C represent only a few of the various ways in which conduits of the invention may be coupled to source of blood (or target vessel), preferably without using suture to facilitate attachment.

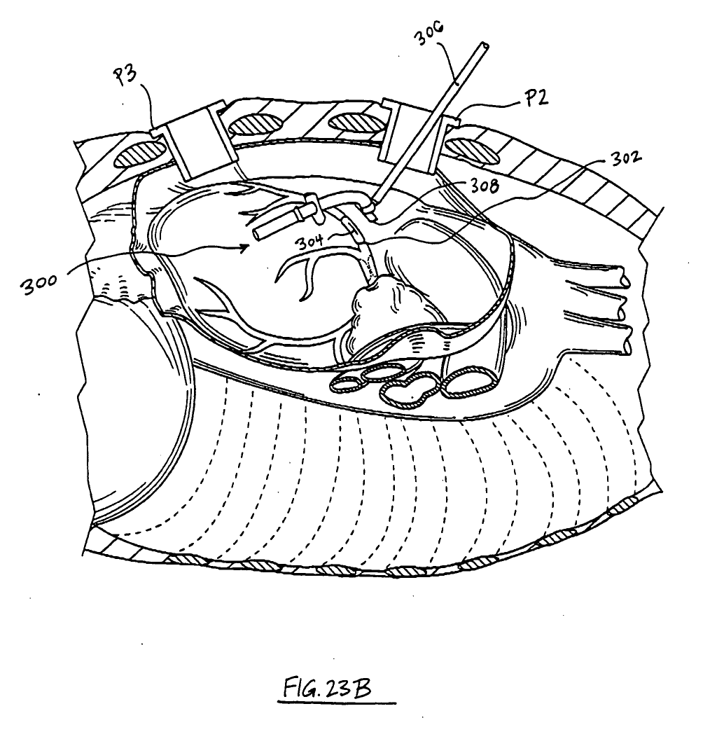

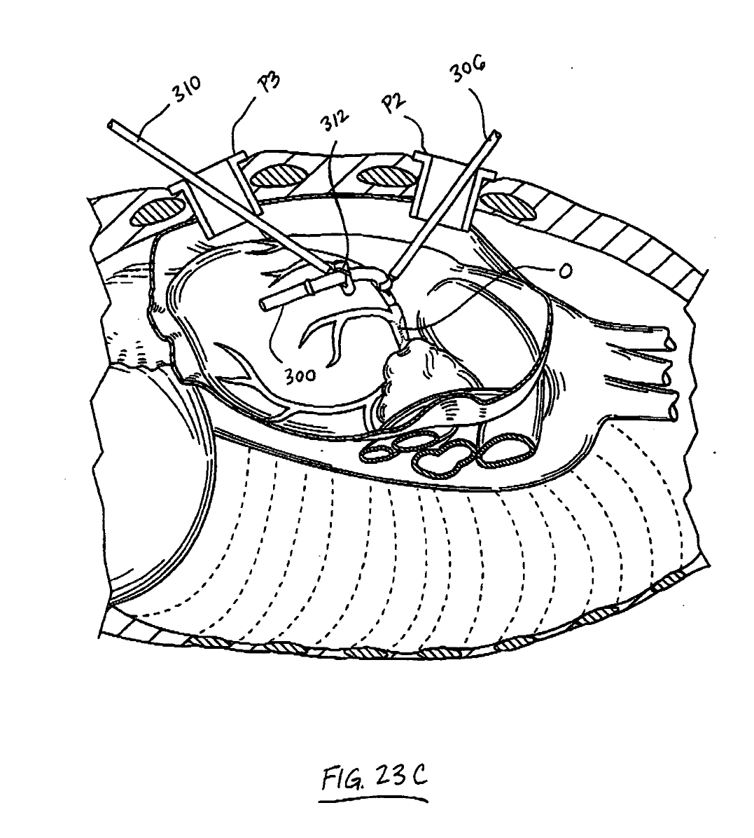

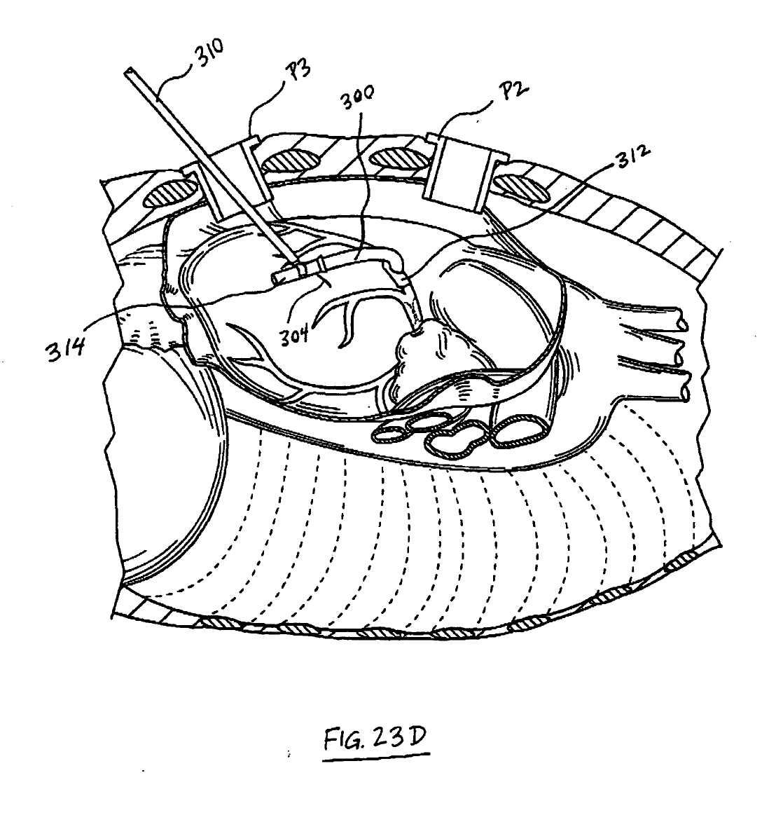

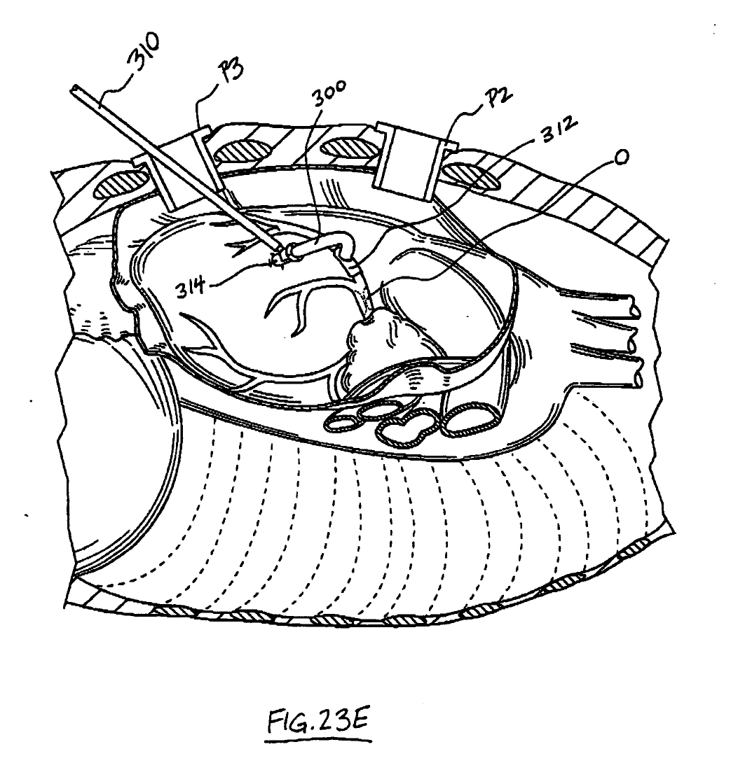

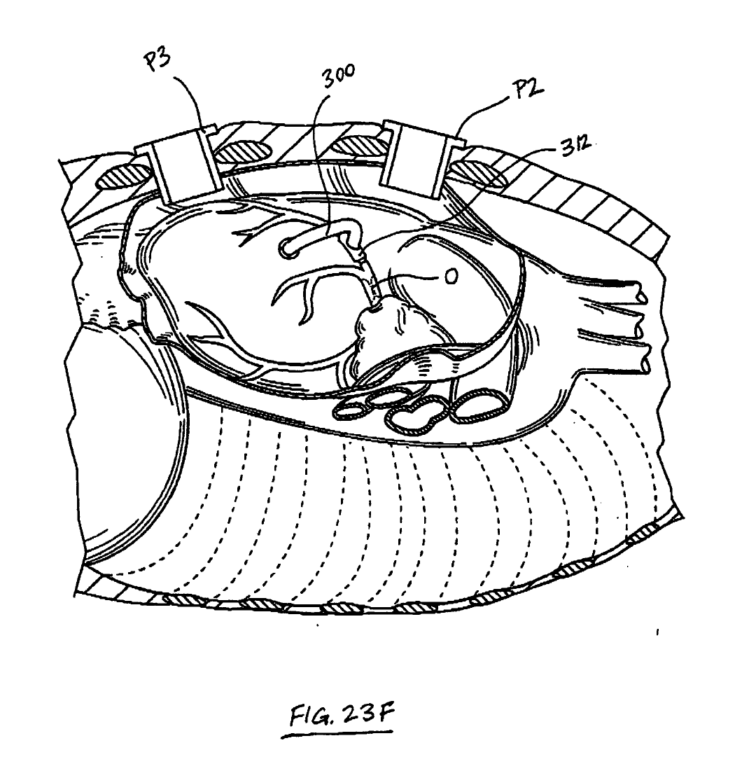

Turning now to FIG. 22 and FIGS. 23A-23F, one possible method of carrying out a ventricular bypass procedure according to the invention will be described. FIG. 22 shows a patient who has been prepared for a minimally invasive surgical procedure by having a plurality of ports P positioned in several intercostal openings. A first port P1 is disposed laterally and may receive a stabilizer that is used to engage and stabilize the work site. For example, if the procedure is carried out on a beating heart the port P1 may receive a stabilizer provided with blower/mister for maintaining a bloodless field. Second and third ports P2 and P3 are used to pass instruments and a conduit constructed according to the invention into the chest cavity. A fourth port P4 is optional and may be used to receive a thoracoscope or other visualization instrument located, for example, at a subxyphoid location.

FIG. 23A is a sectional view of the chest cavity wherein ports P1 and P4 have been omitted for clarity. FIG. 23A shows that the target vessel CA, which has a proximal occlusion O, is located generally under the ports P2 and P3. In use, a stabilizer (not shown) may be introduced via one of the ports and used to maintain the site relatively motionless. FIG. 23B shows a conduit 300 disposed alongside the target vessel CA which has preferably, but not necessarily, been snared at 302. An incision 304 is formed in the target vessel CA, and a delivery device 306 located in port P2 is used to deploy the conduit 300. The delivery device 306 has a working end that is used to place a first securing component 308 of the conduit 300 in the vessel lumen (e.g., as described above with respect to previous embodiments).

FIG. 23C shows another delivery device 310 passed through the port P3 and engaged with a second securing component 312. The delivery device 310 is used to slide the second securing component 312 toward the first securing component 308 to sandwich the vessel wall between the components. Next, as shown in FIG. 23D, the delivery device 310 is used to grasp a transmyocardial portion 314 of the conduit 300 and place the portion 314 into an incision in the myocardium. FIG. 23E shows the transmyocardial portion 314 partially inserted into the myocardial tissue. FIG. 23F shows the conduit fully deployed with the delivery devices 306, 310 removed. The ports P1-P4 are removed from the patient and the intercostal openings are closed.

It will be recognized that practicing minimally invasive procedures according to the invention is not limited to using the specific conduits shown and described herein. As such, although a conduit with an attachment portion including first and second securing components is illustrated, conduits utilizing alternative attachment structure, e.g., stents, clips, staples, suture, etc., may be delivered and deployed according to this embodiment as well.

Further, although the invention is described primarily in connection with cardiovascular applications, it will be understood that the inventive devices and methods are not so limited. For example, the invention may be used to form and deploy an arteriovenous (AV) shunt for use in dialysis treatment. The conduit can be quickly and easily coupled to an artery and vein, and it can be formed of a suitable synthetic vascular graft material capable of withstanding repeated access sticks.

It will also be appreciated that the type of procedure (e.g., open chest, minimally invasive, percutaneous, etc.) used to deploy the conduits of the invention, and thus the accompanying delivery devices, may vary depending on the vessels being treated and user preference. The delivery devices may be relatively short with a substantially rigid shaft assembly for use in open surgical procedures, or they may be longer with a more flexible shaft assembly configured to be guided to a site. In the latter case the device preferably has actuators located near its proximal end to allow remote deployment of the conduit, for example, as disclosed in the aforementioned, co-pending, commonly-owned application Ser. No. 09/304,140. In connection with cardiovascular applications the invention may be used in beating heart procedures, stopped-heart procedures utilizing cardiopulmonary bypass (CPB), or procedures during which the heart is intermittently stopped and started.

As noted above, the conduits of the invention may comprise tissue, synthetic graft material, or a combination of the two; for instance, a saphenous vein graft secured to a conduit attachment portion. The conduit attachment portion also could be coupled to a native artery, such as the left internal mammary artery. For instance, the mammary artery could be taken down and the conduit attachment portion secured to an end thereof as disclosed herein. The artery would then be anastomosed to a coronary artery via the attachment portion.

An inventive conduit may be constructed differently from the configurations specifically illustrated herein. For example, the conduit could be made according to any of the teachings of co-pending, commonly owned application Ser. No. 09/393,131, filed on Sep. 10, 1999 (Attorney Docket No. 010) and entitled “Conduits for Placing a Target Vessel in Fluid Communication With a Source of Blood,” the entire subject matter of which application is incorporated herein by reference.

Moreover, the conduit of the invention may be manufactured by various processes and from various materials; for example, the conduit may be molded (or fabricated from) a material having desired blood interface qualities as well as a desired combination of flexibility and column strength. Manufacturing processes and materials for forming the conduits disclosed herein are disclosed in co-pending, commonly owned application Ser. No. 09/394,119, filed on Sep. 10, 1999 (Attorney Docket No. 011) and entitled “Methods and Devices for Manufacturing a Conduit for Use in Placing a Target Vessel in Fluid Communication With a Source of Blood,” the entire subject matter of which application is incorporated herein by reference.

It may be desirable to utilize a conduit delivery device having a portion surrounding the conduit to restrain and or protect the conduit material prior to and during deployment. The device may have a bore that receives an optional incising element that is extended and retracted, the bore also acting as a flashback lumen to indicate when the device has entered a blood-filled space, for example, a coronary artery or heart chamber.

The conduits of the invention may be provided with a valve or other means for controlling or regulating blood flow. Suitable valves, as well as means for measuring myocardial thickness or verifying entry into the heart chamber, are disclosed in application Ser. No. 09/023,492, filed on Feb. 13, 1998, and entitled “Methods and Devices Providing Transmyocardial Blood Flow to the Arterial Vascular System of the Heart,” the entire subject matter of which has been incorporated herein by reference. The valve could be located at various locations, e.g., the conduit body or the conduit end adapted to communicate with the blood source. Similarly, the conduits may be provided with a reservoir for retaining and discharging blood in a desired manner, the reservoir located at any desired position.

It will be appreciated that the features of the various preferred embodiments of the invention may be used together or separately, while the illustrated methods and devices may be modified or combined in whole or in part. As an example, either of the securing components could be formed as a multipiece or multilayer structure having a desired amount of rigidity or flexibility. Also, more than one conduit may be coupled to a manifold that is placed in communication with one source of blood so as to deliver blood to multiple target vessels. The conduits and devices of the invention may include removable or detachable components, could be formed as disposable instruments, reusable instruments capable of being sterilized, or comprise a combination of disposable and reusable components.

It will be recognized that the invention is not limited to the illustrated applications. For example, an inventive conduit may be coupled to an existing CABG graft that has partially or completely occluded over time by plugging the second conduit portion into the graft distal to the occlusion.

It will be recognized that the invention may be used to manufacture conduits the use of which is not limited to cardiovascular applications such as those illustrated and discussed above. For example, the invention may be used to produce conduits used to carry out many different bypass procedures, including, without limitation, femoral-femoral, femoral-popliteal, femoral-tibial, ilio-femoral, axillary-femoral, subclavian-femoral, aortic-bifemoral, aorto-iliac, aorto-profunda femoris and extra-anatomic.

The conduit may be used to establish fluid communication with many different vessels, including, without limitation, the renal arteries, mesenteric vessel, inferior mesenteric artery, eroneal trunk, peroneal and tibial arteries. Still other applications for the invention include arteriovenous shunts. The conduit may have one, both or more ends configured to engage a target vessel for receiving blood from or delivering blood to another vessel.

The preferred embodiments of the invention are described above in detail for the purpose of setting forth a complete disclosure and for sake of explanation and clarity. It will be readily understood that the scope of the invention defined by the appended claims will encompass numerous changes and modifications.

1-19. (canceled)

20. A device for placing a target vessel in fluid communication with a source of blood, the device comprising:
a conduit adapted to deliver blood from a blood source to a lumen of a target vessel; and
first and second securing components respectively configured to engage inner and outer surfaces of a wall of the target vessel adjacent an incision formed in the target vessel wall, wherein the first and second securing components include a tissue-capturing mechanism that at least partially captures tissue of the target vessel wall;
wherein the conduit is coupled to one of the first and second securing components and is secured to the target vessel wall by the tissue-capturing mechanism;
wherein the tissue-capturing mechanism is configured to substantially fix the relative position of the first and second securing components in the tissue-capturing position without penetrating the target vessel wall tissue other than forming the incision in the target vessel wall.

21. The device of claim 20, wherein the conduit is coupled to the first securing component, and the second securing component has an opening through which the conduit passes, and the opening seals against an exterior surface of the conduit.

22. The device of claim 20, further comprising a mechanism for maintaining the first and second securing components in the tissue-capturing position, wherein the mechanism comprises at least one length of fastening material secured to the first securing component and passing through an aperture in the second securing component, and the length of fastening material is tensioned to fix the relative positions of the first and second securing components.

23. The device of claim 22, wherein a plurality of lengths of fastening material are secured to the first securing component and a plurality of corresponding apertures are formed in the second securing component.

24. The device of claim 23, wherein the fastening material comprises lengths of suture.

25. The device of claim 20, wherein the conduit is formed to assume a low profile with respect to the target vessel and the blood source in use.

26. The device of claim 20, wherein the conduit is reinforced by a coil to prevent the conduit from collapsing during use.

27. The device of claim 26, wherein the coil is a separate member joined to one of the first and second securing components.

28. The device of claim 27, wherein an end of the coil is threaded through openings formed in the first securing component and is fixed thereto.

29. The device of claim 20, wherein at least one of the first and second securing components is generally rectangular with straight sides and at least one rounded end.

30. The device of claim 20, wherein the first securing component comprises a base member with a coating formed of a material selected from the group consisting of silicone, expanded polytetrafluoroethylene, polyurethane, polyamides, polyimides, fluoroethylpolypropylene and polypropylfluorinated amines

31. The device of claim 20, wherein the second securing component is configured to overlie an exterior surface the target vessel wall and is saddle-shaped so as to substantially surround the first securing component.

32. The device of claim 31, wherein the first securing component is configured to lie within at least part of the target vessel lumen and is saddle-shaped so as to substantially match the profile of the second securing component.

33. The device of claim 20, wherein the mechanism for maintaining the first and second securing components in a tissue-capturing position comprises locking elements that are carried by the securing component and snapped together to capture the tissue.

34-69. (canceled)
